(19) United States
(12) Patent Application Publication
(10) Pub. No.: US 2023/0338347 A1
(43) Pub. Date: Oct. 26, 2023

(54) New Use of Inhibitors of the Notch Signalling Pathway

(71) Applicant: Cellestia Biotech AG, Basel (CH)

(72) Inventors: Michael Bauer, Lörrach (DE); Rajwinder Lehal, Zürich (CH); Maximilien Murone, Basel (CH); Pavel Pisa, Gersau (CH); Florian Vogl, Therwil (CH); Maria Bobadilla, Rosenau (FR)

(21) Appl. No.: 18/026,571

(22) PCT Filed: Sep. 16, 2021
(86) PCT No.: PCT/EP2021/075466

§ 371 Date: Mar. 15, 2023

(30) Foreign Application Priority Data
Sep. 16, 2020 (EP) 20196439.2

(51) Int. Cl. A61K 31/4412; A61P 35/00

(52) U.S. Cl. CPC A61K 31/4412; A61P 35/00

(57) ABSTRACT

The present invention relates to new uses and dosage regimens of inhibitors of the Notch signalling pathway such as 6-(4-Tert-Butylphenoxy)Pyridin-3-Amine for the treatment of tumours.

The present invention relates to new uses and dosage regimens of inhibitors of the Notch signalling pathway such as 6-(4-Tert-Butylphenoxy)Pyridin-3-Amine in the treatment of cancers.

### BACKGROUND OF THE INVENTION

The Notch signalling pathway represents a critical component in the molecular circuits that control cell fate during development, cell survival and cell proliferation (Shih IeM, Wang TL in Cancer Res 2007; 67(5):1879-82). Aberrant activation of this pathway contributes to tumourigenesis. The Notch family members are being revealed as oncogenes in an ever-increasing number of cancers. The role of Notch activating mutations and amplification of Notch genes in human cancer and has been highlighted recently and it was demonstrated that genes/proteins in the Notch signalling pathway could be therapeutic targets. Early development attempts focused on the blockage of ligand interaction with Notch receptors through monoclonal antibodies directed against either the ligand or the Notch receptor, and inhibition of the γ-secretase that cleaves the Notch intracellular domain (NICD) from the transmembrane Notch complex, with the common goal of suppressing the aberrant Notch activity. Though significant progress has been made in dissecting the complex workings of this signalling pathway, there are very limited options available for developing novel Notch inhibitors. However, the pioneering class of Notch inhibitors is already in clinical trials for few cancer types, such as γ-secretase inhibitors AL101 from Ayala Pharma (formerly BMS 906024), LY3039478 from Eli Lilly, and Nirogacestat from Springworks Therapeutics, a synthetic small molecule, inhibits the Notch signalling pathway, which may result in induction of growth arrest in tumour cells in which the Notch signalling pathway is overactivated.

One of the drawbacks of use of γ-secretase inhibitors to block Notch signaling, as currently under investigation, is their wide range of additional targets such as amyloid precursor protein. Due to their ability to block Notch signalling via all four receptors γ-secretase inhibitors are known to cause goblet cell metaplasia in the intestine, which may lead to severe diarrhea in patients, in turn prohibiting reaching the necessary drug doses. In addition, some of the hematological malignancies and solid tumours harbor mutations in the Notch receptors (such as chromosomal translocations) resulting in constitutive expression of dominant active form of NICD independent of cleavage by γ-secretase complex. Therefore, these tumours will fail to respond to γ-secretase inhibitors treatment.

Another class of agents under development is the monoclonal antibodies (mAbs) either against Notch receptors (NOTCH1, NOTCH2, NOTCH3) or NOTCH ligands (DLL3, DLL4). Experimental evidence demonstrates that Notch inhibition by either mAb against NOTCH1 or NOTCH2 alleviates the toxicities seen with γ-secretase inhibitors but leads to severe gastrointestinal toxicities (Wu et al. (2010). Therapeutic antibody targeting of individual Notch receptors. Nature 464, 1052-1057).

WO 2013/093885 discloses new inhibitors of the Notch signalling pathway such as 6-(4-Tert-Butylphenoxy)Pyridin-3-Amine which seems useful in cancers where Notch driven cancers are resistant to γ-secretase inhibitor treatment. However, WO 2013/093885 does not disclose whether the inhibitors can be safely used in patients. Therefore, there is still a need for specific and selective treatments of cancers.

### SUMMARY OF THE INVENTION

The object of the present invention is to provide Notch signalling inhibitors for therapeutic use in the treatment of cancer with an improved safety profile and increased efficacy.

Accordingly, in a first aspect, the present invention is directed to a method of treating a Notch-dependent cancer in a subject, comprising administering to said subject 6-(4-Tert-Butylphenoxy)Pyridin-3-Amine or one of its salts, solvates, tautomers, or stereoisomers thereof, wherein about 100 mg to about 700 mg of 6-(4-Tert-Butylphenoxy)Pyridin-3-Amine is administered daily to said subject.

Accordingly, in a further aspect, the present invention is directed to 6-(4-Tert-Butylphenoxy)Pyridin-3-Amine or one of its salts, solvates, tautomers, or stereoisomers thereof for use in the treatment of a Notch-dependent cancer in a subject comprising daily administration of about 100 mg to about 700 mg of 6-(4-Tert-Butylphenoxy)Pyridin-3-Amine to said subject.

Accordingly, in a further aspect, the present invention is directed to the use of 6-(4-Tert-Butylphenoxy)Pyridin-3-Amine or one of its salts, solvates, tautomers, or stereoisomers thereof, for the manufacture of a medicament for treating a Notch-dependent cancer in a subject comprising daily administration of about 100 mg to about 700 mg of 6-(4-Tert-Butylphenoxy)Pyridin-3-Amine to said subject.

In a further aspect, the present invention is directed to a pharmaceutical composition in unit dose comprising 6-(4-Tert-Butylphenoxy)Pyridin-3-Amine or one of its salts, solvates, tautomers, or stereoisomers thereof, wherein the pharmaceutical composition comprises about 100 mg to about 700 mg of 6-(4-Tert-Butylphenoxy)Pyridin-3-Amine, and a pharmaceutically acceptable carrier.

In a further aspect, the present invention is directed to a pharmaceutical composition in unit dose comprising 6-(4-Tert-Butylphenoxy)Pyridin-3-Amine or one of its salts, solvates, tautomers, or stereoisomers thereof, wherein the pharmaceutical composition comprises about 100 mg to about 700 mg of 6-(4-Tert-Butylphenoxy)Pyridin-3-Amine, for use in the treatment of a Notch-dependent cancer in a subject, and a pharmaceutically acceptable carrier.

Further features and advantages of the invention will become apparent from the following description.

### BRIEF DESCRIPTION OF THE FIGURES

FIG. 1 shows swimmer plot by duration of treatment with 6-(4-Tert-Butylphenoxy)Pyridin-3-Amine. Each lane represents one patient and the length of the lane represents the duration of treatment. Best response was durable stable disease at doses showing target engagement. Ten patients with ACC had radiologically confirmed stable disease, of whom 4 patients had a Notch activating mutation. Disease control rate of 19 patients with adenoid cystic carcinoma at 8 weeks and 20 weeks was 79% and 58%, respectively (calculated as number of patients with complete or partial response, or stable disease divided by the number of patients with that indication treated). Abbreviations: SD, stable disease; PD, progressive disease; NA, not applicable).

FIG. 2 shows time-dependent downregulation of DTX-1 (Deltex E3 Ubiquitin Ligase 1). DTX-1 is a positive regulator of NOTCH signalling pathway (C1D01_pre: Cycle 1 Day 1 predose; C1D01_1 h: Cycle 1 Day 1 1 hour post dose; C1D21_1 h: Cycle 1 Day 21 1 hour post dose; C2D15_1 h: Cycle 2 Day 15 1 hour post dose).

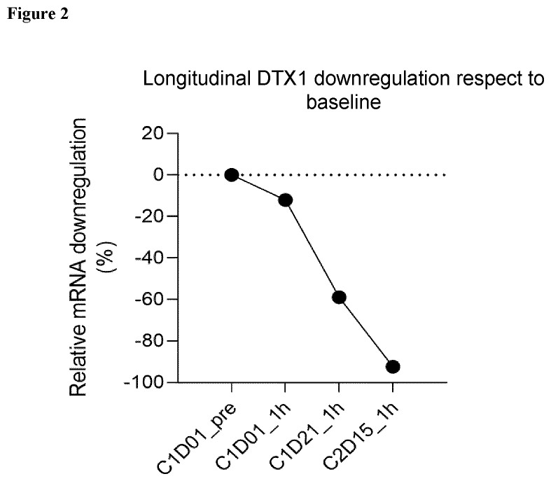

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. In addition, the materials, methods, and examples are illustrative only and are not intended to be limiting.

In case of conflict, the present specification including the definitions therein will prevail. Unless defined otherwise, all technical and scientific terms used herein will have the same meaning as commonly understood by one of skill in the art to which the subject-matter herein belongs. As used herein, the following definitions are supplied in order to facilitate the understanding of the present invention.

As used herein, the term “comprise/comprising” is generally used in the sense of include/including, i.e. permitting the presence of one or more features or components. The terms “comprise” and “comprising” also encompass the more restricted terms “consist” and “consisting”.

As used herein, the singular form “a”, “an” and “the” include plural references unless the context clearly dictates otherwise.

As used herein, the term “administering” in relation to a compound, e.g., 6-(4-Tert-Butylphenoxy)Pyridin-3-Amine or a standard of care agent, is used to refer to delivery of that compound by any route of delivery.

As used herein, the terms “daily administration”, “administered daily” or “daily dose”, mean the administration or dose of a compound, e.g. 6-(4-Tert-Butylphenoxy)Pyridin-3-Amine, a subject receives per day. Daily administration or daily dose can be once daily, twice daily or even more frequently per day. The compound is administered every day but administration may be paused at certain intervals (eg, given every week on 5 consecutive days followed by two days without taking the compound).

As used herein, the term “pharmaceutically acceptable carrier” means a carrier or excipient that is useful in preparing a pharmaceutical composition that is generally safe, and possesses acceptable toxicities. Acceptable carriers include those that are acceptable for veterinary use as well as human pharmaceutical use. A “pharmaceutically acceptable carrier” as used in the specification and claims includes both one and more than one such carrier.

As used herein, the term “about” in relation to a numerical value x means, for example, +/−10%.

As used herein, the word “substantially” does not exclude “completely,” e.g., a composition which is “substantially free” from Y may be completely free from Y.

As used herein, the terms “patient” or “subject” is well-recognized in the art and can be used interchangeably and is preferably a human subject. They can refer to a mammal, including dog, cat, rat, mouse, monkey, cow, horse, goat, sheep, pig, camel, and, most preferably, a human. In some embodiments, the subject is a subject in need of treatment or a subject with a disease or disorder, such as cancer. However, in other embodiments, the subject can be a normal subject or a subject who has already undergone a treatment against cancer. The term does not denote a particular age or sex. Thus, all male or female adults, adolescents, children and newborn subjects, are intended to be covered.

The terms “tumour”, “cancer”, “malignancy”, “cancer cells”, “cell proliferative diseases” and “cell proliferative disorders” as used herein refer to or describe the physiological condition in mammals that is typically characterized by unregulated cell growth and includes advanced tumour and advanced cancer like advanced metastatic tumour and advanced metastatic cancer. Unless defined otherwise herein “tumour”, “cancer”, “malignancy”, “cancer cells”, “cell proliferative diseases” and “cell proliferative disorders” as used herein are Notch-dependent.

The term “malignant solid tumour” or “malignant solid tumour indication” used herein refers to an abnormal mass of tissue characterized by uncontrolled cell proliferation. Malignant solid tumours are treated with the methods of the present invention. Different types of malignant solid tumours are named for the type of cells that form them. Examples of malignant solid tumours are sarcomas, carcinomas, and lymphomas. Leukemias (cancers of the blood) generally do not form malignant solid tumours (definition according to the national cancer institute of the NIH). Malignant solid tumours include, but are not limited to, abnormal mass of cells which may stem from different tissue types such as liver, colon, colorectum, skin, breast, pancreas, cervix uteri, corpus uteri, bladder, gallbladder, kidney, larynx, lip, oral cavity, oesophagus, ovary, prostate, stomach, testis, thyroid gland or lung.

“Hematologic malignancies” are cancers that affect the blood, bone marrow, and lymph nodes. Hematologic malignancies are treated with the methods of the present invention. This classification includes but are not limited to various types of leukemia (acute lymphocytic leukemie, chronic lymphocytic leukemia, acute myeloid leukemia, chronic myeloid leukemia), myeloma, and lymphoma (Hodgkin's and non-Hodgkin's).

“Advanced tumour”, “advanced cancer” “advanced metastatic tumour”, “advanced metastatic cancer” or “advanced malignancy” means that the cancer or tumour may have spread from where it first started to nearby tissue, lymph nodes, or distant parts of the body. Preferably advanced metastatic tumour and/or advanced metastatic cancer are treated with the methods of the present invention. Treatment may be given to help shrink the tumour, slow the growth of cancer cells, or relieve symptoms. Optionally the advanced tumour/cancer has not responded to standard of care.

The term “Notch-dependent” or “Notch-dependent cancer” as used herein refers to a Notch signaling pathway activated cancer. The Notch signaling pathway is one of the most commonly activated signaling pathways in cancer. A “Notch-dependent cancer” can have one or more of the following genetic causes:
Chromosomal translocation overlapping with NOTCH regions on the chromosomes, leading to fusion of NOTCH related genes and expression of truncated versions of the NOTCH 1,2, 3 and/or 4 proteins, causing ligand-/receptor-independent constitutive activation_of the NOTCH pathway.
Gain of function (GOF) mutations in the NOTCH genes leading to a constitutive, ligand-/receptor-independent activation of the Notch pathway. Loss of function (LOF) mutations in negative regulators of the Notch pathway, like FBXW7 or NUMB.
Over-expression of the different Notch-specific ligands or receptors, leading to extended activation of the NOTCH pathway.

More preferably according to the present invention, cancers are Notch-dependent cancers that can be either solid tumours or hematological malignancies. In one embodiment, the Notch-dependent cancer is resistant to γ-secretase inhibitor treatment. Examples of γ-secretase inhibitor treatment comprise 1) Gamma secretase inhibitor RO4929097 and Cediranib Maleate in treating patients with advanced solid tumours (NCT01131234), 2) Gamma-Secretase Inhibitor RO4929097 in Treating Young Patients With Relapsed or Refractory Solid Tumours, CNS Tumours, Lymphoma, or T-Cell Leukemia (NCT01088763), 3) Study of MK-0752 in combination with Tamoxifen or Letrozole to treat early stage breast cancer (NCT00756717), 4) GDC-0449 and RO4929097 in treating patients with Advances or metastatic sarcoma (NCT01154452) 5) RO4929097 and Erlotinib Hydrochloride in treating patients with stage IV or recurrent Non-Small Cell Lung Cancer (NCT01193881), 6) Bicalutamide and RO4929097 in treating patients with previously treated prostate cancer (NCT01200810), 7) RO4929097 in treating patients with recurrent invasive Gliomas (NCT01269411), 8) A Notch signaling pathway inhibitor for patients with T-cell Acute Lymphoblastic Leukemia/Lymphoma (ALL) (NCT00100152) and 9) RO4929097 in treating patients with metastatic colorectal cancer (NCT01116687).

“6-(4-Tert-Butylphenoxy)Pyridin-3-Amine” denotes a compound according to Formula I below. 6-(4-Tert-Butylphenoxy)Pyridin-3-Amine is a synthetic small molecule (Molecular Mass: 242.32 g/mol).

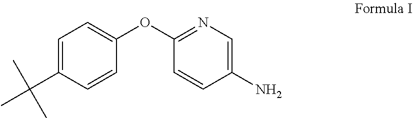 [SMILES: CC(C)(C)c1ccc(Oc2ccc(N)cn2)cc1]

“6-(4-Tert-Butylphenoxy)Pyridin-3-Amine monohydrochloride” or “6-(4-Tert-Butylphenoxy)Pyridin-3-Amine monohydrochloride salt” denotes the monohydrochloride salt of the compound according to Formula I above (Molecular Mass: 278.78 g/mol).

Patients with advanced and metastatic tumours often have limited therapeutic options beyond the accepted standard of care. For patients, whose cancer has become refractory to available treatments, there is a significant unmet therapeutic need. For certain tumors there is no approved treatment available and no standard of care exists, as for example in Adenoid Cystic Carcinoma. In some malignancies, the cancer pathology can be driven by, or be largely dependent, on well-defined genetic aberrations in selected cellular signalling pathways like mutations, chromosomal translocations or overexpression of the respective ligands or receptors.

Over-activation of the NOTCH signalling pathway, in particular the constitutive activation of NOTCH signalling (independent of ligand/receptor expression) in certain cancer indications is correlated with a more aggressive course of disease, resulting in poorer survival rates with a more rapid disease progression compared to the overall survival (OS) seen in patients with the same tumours not having any aberration or dysregulation of the NOTCH pathway.

Based on the clinical studies disclosed in the experimental section herein, it has now been found that 6-(4-Tert-Butylphenoxy)Pyridin-3-Amine provides clinical benefit and an improved safety profile in the treatment of NOTCH-dependent cancers. An “improved safety profile” as referred herein means that the safety profile of 6-(4-Tert-Butylphenoxy)Pyridin-3-Amine is improved compared to other drugs targeting the Notch signalling pathway.

Therefore, in a first aspect of the invention, a method of treating a Notch-dependent cancer in a subject is provided, comprising administering to said subject 6-(4-Tert-Butylphenoxy)Pyridin-3-Amine or one of its salts, solvates, tautomers, or stereoisomers thereof, wherein about 50 mg to about 700 mg, preferably about 100 mg to about 700 mg, of 6-(4-Tert-Butylphenoxy)Pyridin-3-Amine is administered daily to said subject.

In one embodiment, 6-(4-Tert-Butylphenoxy)Pyridin-3-Amine is a hydrochloride salt or a hydrobromide salt, preferably a hydrochloride salt. In a more preferred embodiment, the 6-(4-Tert-Butylphenoxy)Pyridin-3-Amine monohydrochloride salt is used in the present invention.

In one embodiment, the cancer is characterised by activation of the NOTCH signalling pathway optionally characterised by alterations and overactivation of the NOTCH signalling pathway e.g. via genetic alterations such as mutations, translocations, and/or over-expression detectable by appropriate laboratory techniques.

In one embodiment, the subject has a solid tumour such as sarcoma or carcinoma. The solid tumour can be confirmed by histological or cytological methods. In some embodiment the solid tumour is advanced and/or metastatic tumour. In some embodiment the solid tumour is advanced and/or metastatic tumour after standard of care treatment. In certain embodiments, the solid tumour is advanced. The cancer may have spread from where it first started to nearby tissue, lymph nodes, or distant parts of the body. Treatment may be given to help shrink the tumour, slow the growth of cancer cells, or relieve symptoms. And in yet another embodiment, the solid tumour has relapsed or progressed upon or is refractory to standard therapy. In another embodiment, the subject has histologically or cytologically confirmed solid tumours that are surgically unresectable, locally advanced, or metastatic which have progressed on at least one line of systemic therapy.

In one embodiment the subject has a cancer selected from the group consisting of adenoid cystic carcinoma (ACC), breast cancer, prostate cancer, osteosarcoma, malignant glomus tumour, colorectal cancer and hepatocellular carcinoma, preferably selected from the group consisting of adenoid cystic carcinoma (ACC), Triple negative breast cancer (TNBC), ER+breast cancer, ER− breast cancer, HER2+ breast cancer, HER2− breast cancer, prostate cancer, osteosarcoma, malignant glomus tumour, colorectal cancer in patients resistant to oxaliplatin or irinotecan-based therapy and hepatocellular carcinoma.

In a preferred embodiment the subject has a cancer selected from the group consisting of adenoid cystic carcinoma (ACC), breast cancer, colorectal cancer and prostate cancer. Breast cancer as referred herein comprises hormone receptor positive or negative breast cancers, such as Estrogen receptor positive (ER+), Estrogen receptor negative (ER−), Progesteron receptor positive (PR+), Progesteron receptor negative (PR−), human epidermal growth factor receptor 2 positive (HER2+) and human epidermal growth factor receptor 2 negative (HER2−) breast cancers. The breast cancer can be a triple negative breast cancer (TNBC), where the cancer does not have receptors for either HER2 or the hormones estrogen and progesterone. In some embodiments the breast cancer is selected from the group consisting of triple negative breast cancer, ER− breast cancer, and HER2+ breast cancer. Colorectal cancer as referred herein comprises colorectal cancer wherein the subject is not resistant to oxaliplatin or irinotecan-based therapy and colorectal cancer wherein the subject is resistant to oxaliplatin or irinotecan-based therapy. In a preferred embodiment the colorectal cancer is colorectal cancer wherein the subject is resistant to oxaliplatin or irinotecan-based therapy.

In a particular embodiment, the subject has adenoid cystic carcinoma (ACC). In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating adenoid cystic carcinoma in a subject is provided, comprising administration of 6-(4-Tert-Butylphenoxy)Pyridin-3-Amine or one of its salts, solvates, tautomers, or stereoisomers thereof, to said subject, preferably to a subject who did not receive prior systemic treatment, in particular did not receive other anti-cancer drugs before treatment with 6-(4-Tert-Butylphenoxy)Pyridin-3-Amine.

In a further particular embodiment, the subject has breast cancer, preferably breast cancer selected from the group consisting of triple negative breast cancer, ER− breast cancer, and HER2+ breast cancer. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating breast cancer, preferably breast cancer selected from the group consisting of triple negative breast cancer, ER− breast cancer, and HER2+ breast cancer, in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In a further particular embodiment, the subject has colorectal cancer, preferably colorectal cancer wherein the subject is resistant to oxaliplatin or irinotecan-based therapy. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating colorectal cancer, preferably colorectal cancer wherein the subject is resistant to oxaliplatin or irinotecan-based therapy. in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In a further particular embodiment, the subject has prostate cancer. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating prostate cancer in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In one embodiment the subject has hepatocellular carcinoma. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating hepatocellular carcinoma in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment

In one embodiment the subject has osteosarcoma. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating osteosarcoma in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In one embodiment the subject has a malignant glomus tumour. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating malignant glomus tumour in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In another embodiment, the subject has sarcoma such as osteosarcoma, liposarcoma, rhabdomyosarcoma, or fibrosarcoma. In other embodiments, the subject has gastric cancer, cholangiocellular carcinoma, ovarian cancer, cervical cancer, prostate cancer, non-small cell lung cancer (NSCLC) and especially lung adenocarcinoma which is subgroup of NSCLC, melanoma, or glioblastoma multiforme.

In one embodiment, the subject has osteosarcoma, liposarcoma, rhabdomyosarcoma, or fibrosarcoma, gastric cancer, cholangiocellular carcinoma, ovarian cancer, cervical cancer, prostate cancer, non-small cell lung cancer, lung adenocarcinoma, melanoma, or glioblastoma multiforme.

In another embodiment, the subject has a haematological malignancy. Haematological malignancies can be confirmed by histological or cytological methods. In certain embodiments, the haematological malignancies are advanced. In yet another embodiment, the haematological malignancy has relapsed or progressed upon or is refractory to standard therapy.

In one embodiment the cancer is selected from the group consisting of acute lymphoblastic leukemia, T-cell acute lymphoblastic leukemia (T-ALL) or T-cell lymphoblastic lymphoma (T-LBL), acute myeloid leukemia, chronic lymphocytic leukemia, chronic myelocytic leukemia, Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma.

Thus in one embodiment, the subject has acute lymphoblastic leukemia, T-cell acute lymphoblastic leukemia (T-ALL) or T-cell lymphoblastic lymphoma (T-LBL), acute myeloid leukemia, chronic lymphocytic leukemia, chronic myelocytic leukemia, Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma. In one embodiment, the subject has acute lymphoblastic leukemia, T-cell acute lymphoblastic leukemia (T-ALL) or T-cell lymphoblastic lymphoma (T-LBL), acute myeloid leukemia, chronic lymphocytic leukemia, or chronic myelocytic leukemia of any cell lineage. In another embodiment, the subject has Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma. Thus in a further particular embodiment, the subject has T-cell acute lymphoblastic leukemia (T-ALL).

The multiple myeloma can be relapsed/refractory to at least two lines of therapy, including both a proteasome inhibitor and an immunomodulatory agent. The Hodgkin lymphoma can be relapsed/refractory to at least two lines of treatment including standard of care such as for instance brentuximab vedotin. The B-cell NHL can be relapsed/refractory upon at least one line of standard treatment such as chemo-immunotherapy. The T-cell NHL can be relapsed/refractory upon at least one line of standard treatment such as chemotherapy. The subject with multiple myeloma can have measurable disease (serum M-protein≥10 g/L or urine M-protein≥200 mg/24 hours or abnormal free light chain (FLC) ratio with involved FLC>100 mg/L) or proven plasmacytoma by biopsy). T-cell acute lymphoblastic leukaemia (T-ALL) or T-cell lymphoblastic lymphoma (T-LBL) can be relapsed or refractory (r/r). Refractory patients in this regard are defined as T-ALL/T-LBL patients with ≥5% bone marrow blasts, and/or concomitant extramedullary involvement, who have not achieved a CR after standard induction/consolidation therapy attempt. Relapsed patients are defined as T-ALL/T-LBL patients who have recurrent disease, i.e. ≥5% bone marrow blasts and/or concomitant extramedullary relapse, after having achieved a prior CR.

In one embodiment, the method comprises daily administration of about 100 mg to about 700 mg of 6-(4-Tert-Butylphenoxy)Pyridin-3-Amine. In one embodiment, the method comprises daily administration of about 150 mg to about 700 mg of 6-(4-Tert-Butylphenoxy)Pyridin-3-Amine. In one embodiment, the method comprises daily administration of about 200 mg to about 700 mg of 6-(4-Tert-Butylphenoxy)Pyridin-3-Amine . In one embodiment, the method comprises daily administration of about 350 mg to about 700 mg of 6-(4-Tert-Butylphenoxy)Pyridin-3-Amine. In one embodiment, the method comprises daily administration of about 500 mg to about 700 mg of 6-(4-Tert-Butylphenoxy)Pyridin-3-Amine. In other embodiments, the daily dose administered to said subject of 6-(4-Tert-Butylphenoxy)Pyridin-3-Amine is about 50 mg to about 100 mg, about 50 mg to about 150 mg, about 50 mg to about 200 mg, about 50 mg to about 350 mg, about 50 mg to about 550 mg, or about 50 mg to about 700 mg. In other embodiments, the daily dose administered to said subject of 6-(4-Tert-Butylphenoxy)Pyridin-3-Amine is about 100 mg to about 200 mg, about 100 mg to about 350 mg, about 100 mg to about 550 mg, or about 100 mg to about 700 mg. In other embodiments, the daily dose administered to said subject of 6-(4-Tert-Butylphenoxy)Pyridin-3-Amine is about 100 mg to about 250 mg, about 200 mg to about 350 mg, about 250 mg to about 450 mg, about 350 mg to about 550 mg, about 400 mg to about 650 mg, or about 500 mg to about 700 mg. In a preferred embodiment the daily dose administered to said subject of 6-(4-Tert-Butylphenoxy)Pyridin-3-Amine is about 50 mg to about 550 mg. In a more preferred embodiment the daily dose administered to said subject of 6-(4-Tert-Butylphenoxy)Pyridin-3-Amine is about 100 mg to about 550 mg. In a further more preferred embodiment the daily dose administered to said subject of 6-(4-Tert-Butylphenoxy)Pyridin-3-Amine is about 100 mg to about 900 mg or about 100 mg to about 1100 mg. In an even more preferred embodiment the daily dose administered to said subject of 6-(4-Tert-Butylphenoxy)Pyridin-3-Amine is about 300 mg to about 700 mg. In a further even more preferred embodiment the daily dose administered to said subject of 6-(4-Tert-Butylphenoxy)Pyridin-3-Amine is about 400 mg to about 700 mg. In a further even more preferred embodiment the daily dose administered to said subject of 6-(4-Tert-Butylphenoxy)Pyridin-3-Amine is about 500 mg to about 900 mg or about 500 mg to about 1100 mg. Most preferred is a daily dose of about 400 mg to about 650 mg of 6-(4-Tert-Butylphenoxy)Pyridin-3-Amine, in particular a daily dose of about 500 mg of 6-(4-Tert-Butylphenoxy)Pyridin-3-Amine, administered to said subject. More particular a daily dose of about 800 mg of 6-(4-Tert-Butylphenoxy)Pyridin-3-Amine is administered to said subject. Even more particular a daily dose of about 1000 mg of 6-(4-Tert-Butylphenoxy)Pyridin-3-Amine is administered to said subject.

In some embodiments, the daily dose is about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of 6-(4-Tert-Butylphenoxy)Pyridin-3-Amine per day. In other embodiments, the daily dose is about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, or 1070-1100 mg. In other embodiments, the daily dose is about 100-110, 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, or 1040-1070 mg.In other embodiments, the daily dose is about 100-110, 120-140, 140-160, 170-180, 200-230, 250-270, 290-320, 340-360, 380-400, 440-460, 470-490, 500-550, 550-570, 590-620, 640-660, or 690-710 mg of 6-(4-Tert-Butylphenoxy)Pyridin-3-Amine. In other embodiments, the daily dose is about 45-55, 100-110, 140-160, 200-230, 340-360, or 500-550, or 690-710 mg, or about 45-55, 100-110, 140-160, 200-230, 340-360, or 500-550 of 6-(4-Tert-Butylphenoxy)Pyridin-3-Amine. In other embodiments, the daily dose is about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg. In other embodiments, the daily dose is about 100, 120, 150, 170, 200, 250, 300, 350, 400, 450, 500, 550, 600, 650, 700, 750, or 800 mg. In other embodiments, the daily dose is about 120, 150, 170, 200, 250, 300, 350, 400, 450, 500, 550, 600, 650, 700, 750, or 800 mg. In other embodiments, the daily dose is about 60, 120, 170, 250, 400, or 600 mg.

In some embodiments, the dosing can also be weight-based and especially for children and adolescents. The dose can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg. The dose can be about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of 6-(4-Tert-Butylphenoxy)Pyridin-3-Amine. The dose can be about 100-110, 120-140, 140-160, 170-180, 200-230, 250-270, 290-320, 340-360, 380-400, 440-460, 470-490, 500-550, 550-570, 590-620, 640-660, or 690-710 mg of 6-(4-Tert-Butylphenoxy)Pyridin-3-Amine per 70 kg patient, the exact dose is then recalculated for a patient having a lower or higher body weight than 70 kg. In another embodiment, the dose can be about 45-55, 100-110, 140-160, 200-230, 340-360, 500-550, or 690-710 or about 45-55, 100-110, 140-160, 200-230, 340-360, or 500-550 mg of 6-(4-Tert-Butylphenoxy)Pyridin-3-Amine per 70 kg patient. In another embodiment, the dose can be about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg per 70 kg patient. In other embodiments, the daily dose is about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, or 1070-1100 mg per 70 kg patient. In other embodiments, the daily dose is about 100-110, 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, or 1040-1070 mg per 70 kg patient. In other embodiments, the daily dose is about 100, 120, 150, 170, 200, 250, 300, 350, 400, 450, 500, 550, 600, 650, 700, 750, or 800 mg per 70 kg patient. In another embodiment, the dose can be about 120, 150, 170, 200, 250, 300, 350, 400, 450, 500, 550, 600, 650, 700, 750, or 800 mg. In another embodiment, the dose can be about about 60, 120, 170, 250, 400, or 600 mg. The exact dose is then recalculated for a patient having a lower or higher body weight than 70 kg, or be calcalulated based on body surface area (BSA).

In one embodiment, the method comprises daily administration of about 120 mg to about 800 mg of the 6-(4-Tert-Butylphenoxy)Pyridin-3-Amine monohydrochloride salt. In one embodiment, the method comprises daily administration of about 170 mg to about 800 mg of the 6-(4-Tert-Butylphenoxy)Pyridin-3-Amine monohydrochloride salt. In one embodiment, the method comprises daily administration of about 250 mg to about 800 mg of the 6-(4-Tert-Butylphenoxy)Pyridin-3-Amine monohydrochloride salt. In one embodiment, the method comprises daily administration of about 400 mg to about 800 mg of the 6-(4-Tert-Butylphenoxy)Pyridin-3-Amine monohydrochloride salt. In one embodiment, the method comprises daily administration of about 600 mg to about 800 mg of the 6-(4-Tert-Butylphenoxy)Pyridin-3-Amine monohydrochloride salt. In other embodiments, the daily dose administered to said subject of the 6-(4-Tert-Butylphenoxy)Pyridin-3-Amine monohydrochloride salt is about 60 mg to about 120 mg, about 60 mg to about 170 mg, about 60 mg to about 250 mg, about 60 mg to about 400 mg, about 60 mg to about 600 mg, or about 60 mg to about 800 mg. In other embodiments, the daily dose administered to said subject of the 6-(4-Tert-Butylphenoxy)Pyridin-3-Amine monohydrochloride salt is about 120 mg to about 250 mg, about 120 mg to about 400 mg, about 120 mg to about 600 mg, or about 120 mg to about 800 mg. In other embodiments, the daily dose administered to said subject of the 6-(4-Tert-Butylphenoxy)Pyridin-3-Amine monohydrochloride salt is about 100 mg to about 300mg, about 200 mg to about 400 mg, about 300 mg to about 500 mg, about 400 mg to about 600 mg, about 500 mg to about 700 mg, or about 600 mg to about 800 mg, wherein 500 mg to about 700 mg is preferred . In a further preferred embodiment the daily dose administered to said subject of the 6-(4-Tert-Butylphenoxy)Pyridin-3-Amine monohydrochloride salt is about 60 mg to about 600 mg. In a more preferred embodiment the daily dose administered to said subject of the 6-(4-Tert-Butylphenoxy)Pyridin-3-Amine monohydrochloride salt is about 120 mg to about 600 mg. In a further more preferred embodiment the daily dose administered to said subject of the 6-(4-Tert-Butylphenoxy)Pyridin-3-Amine monohydrochloride salt is about 120 mg to about 1000 mg or about 120 mg to about 1200 mg. In an even more preferred embodiment the daily dose administered to said subject of the 6-(4-Tert-Butylphenoxy)Pyridin-3-Amine monohydrochloride salt is about 120 mg to about 800 mg. In a further even more preferred embodiment the daily dose administered to said subject of the 6-(4-Tert-Butylphenoxy)Pyridin-3-Amine monohydrochloride salt is about 600 mg to about 1000 mg or about 600 mg to about 1200 mg. Most preferred is a daily dose of about 500 mg to about 700 mg of the 6-(4-Tert-Butylphenoxy)Pyridin-3-Amine monohydrochloride salt, in particular about 600 mg of the 6-(4-Tert-Butylphenoxy)Pyridin-3-Amine monohydrochloride salt, administered to said subject. More particular a daily dose of about 900 mg of the 6-(4-Tert-Butylphenoxy)Pyridin-3-Amine monohydrochloride salt is administered to said subject. Even more particular a daily dose of about 1150 mg of the 6-(4-Tert-Butylphenoxy)Pyridin-3-Amine monohydrochloride salt is administered to said subject.

In some embodiments, the daily dose is about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg of the 6-(4-Tert-Butylphenoxy)Pyridin-3-Amine monohydrochloride salt. In other embodiments, the daily dose is about 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of the 6-(4-Tert-Butylphenoxy)Pyridin-3-Amine monohydrochloride salt. In other embodiments, the daily dose is about 120-140, 140-160, 170-180, 200-230, 250-270, 290-320, 340-360, 380-400, 440-460, 470-490, 500-550, 550-570, 590-620, 640-660, or 690-710 mg of the 6-(4-Tert-Butylphenoxy)Pyridin-3-Amine monohydrochloride salt. In other embodiments, the daily dose is about120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, 1070-1100, 1100-1130, 1130-1160, or 1160-1200 mg of the 6-(4-Tert-Butylphenoxy)Pyridin-3-Amine monohydrochloride salt. In other embodiments, the daily dose is about about 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, 1040-1070, 1100-1130, or 1160-1200 mg of the 6-(4-Tert-Butylphenoxy)Pyridin-3-Amine monohydrochloride salt. In other embodiments, the daily dose is about 120, 150, 170, 200, 250, 300, 350, 400, 450, 500, 550, 600, 650, 700, 750, or 800 mg of the 6-(4-Tert-Butylphenoxy)Pyridin-3-Amine monohydrochloride salt. In other embodiments, the daily dose is about 60, 120, 170, 250, 400, or 600 mg of the 6-(4-Tert-Butylphenoxy)Pyridin-3-Amine monohydrochloride salt.

In some embodiments, the dosing can also be weight-based and especially for children and adolescents. The dose can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg. The dose can be 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780 or 800 mg of the 6-(4-Tert-Butylphenoxy)Pyridin-3-Amine monohydrochloride salt per 70 kg patient, the exact dose is then recalculated for a patient having a lower or higher body weight than 70 kg. In another embodiment, the dose can be 60, 120, 170, 250, 400, 600, 700, or 800 mg of the 6-(4-Tert-Butylphenoxy)Pyridin-3-Amine monohydrochloride salt per 70 kg patient, the exact dose is then recalculated for a patient having a lower or higher body weight than 70 kg, or be calcalulated based on body surface area (BSA). In other embodiments, the daily dose is about 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of the 6-(4-Tert-Butylphenoxy)Pyridin-3-Amine monohydrochloride salt per 70 kg patient. In other embodiments, the daily dose is about 120-140, 140-160, 170-180, 200-230, 250-270, 290-320, 340-360, 380-400, 440-460, 470-490, 500-550, 550-570, 590-620, 640-660, or 690-710 mg of the 6-(4-Tert-Butylphenoxy)Pyridin-3-Amine monohydrochloride salt per 70 kg patient. In other embodiments, the daily dose is about120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, 1070-1100, 1100-1130, 1130-1160, or 1160-1200 mg of the 6-(4-Tert-Butylphenoxy)Pyridin-3-Amine monohydrochloride salt per 70 kg patient. In other embodiments, the daily dose is about about 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, 1040-1070, 1100-1130, or 1160-1200 mg of the 6-(4-Tert-Butylphenoxy)Pyridin-3-Amine monohydrochloride salt per 70 kg patient.

In one embodiment, 6-(4-Tert-Butylphenoxy)Pyridin-3-Amine is administered twice a day. The period between two administrations per day is usually 8-12 hours.

Thus in one embodiment, the dose administered to said subject of 6-(4-Tert-Butylphenoxy)Pyridin-3-Amine twice a day is about 12.5 mg to about 350 mg. In one embodiment, the dose administered to said subject of 6-(4-Tert-Butylphenoxy)Pyridin-3-Amine twice a day is about 25 mg to about 350 mg. In one embodiment, the dose administered to said subject of 6-(4-Tert-Butylphenoxy)Pyridin-3-Amine twice a day is about 75 mg to about 350 mg. In one embodiment, the dose administered to said subject of 6-(4-Tert-Butylphenoxy)Pyridin-3-Amine twice a day is about 100 mg to about 350 mg. In one embodiment, the dose administered to said subject of 6-(4-Tert-Butylphenoxy)Pyridin-3-Amine twice a day is about 150 mg to about 350 mg. In one embodiment, the dose administered to said subject of 6-(4-Tert-Butylphenoxy)Pyridin-3-Amine twice a day is about 250 mg to about 350 mg.

In other embodiments, the dose administered to said subject of 6-(4-Tert-Butylphenoxy)Pyridin-3-Amine twice a day is about 12.5 mg to about 25 mg, about 12.5 mg to about 50 mg, about 12.5 mg to about 75 mg, about 12.5 mg to about 100 mg, about 12.5 mg to about 175 mg, about 12.5 mg to about 250 mg, or about 12.5 mg to about 350 mg. In other embodiments, the dose administered to said subject of 6-(4-Tert-Butylphenoxy)Pyridin-3-Amine twice a day is about 25 mg to about 50 mg, about 25 mg to about 75 mg, about 25 mg to about 100 mg, about 25 mg to about 150 mg, or about 25 mg to about 200 mg. In other embodiments, the dose administered to said subject of 6-(4-Tert-Butylphenoxy)Pyridin-3-Amine twice a day is about 50 mg to about 150 mg, about 100 mg to about 200 mg, about 100 mg to about 250 mg, about 200 mg to about 250 mg, about 200 mg to about 300 mg, or about 250 mg to about 350 mg. In a further preferred embodiment the dose administered to said subject of 6-(4-Tert-Butylphenoxy)Pyridin-3-Amine twice a day is about 12.5 mg to about 250 mg. In a further preferred embodiment the dose administered to said subject of 6-(4-Tert-Butylphenoxy)Pyridin-3-Amine twice a day is about 25 mg to about 250 mg. In a further preferred embodiment the dose administered to said subject of 6-(4-Tert-Butylphenoxy)Pyridin-3-Amine twice a day is about 150 mg to about 350 mg. Particular preferred is a dose of about 200 mg to about 350 mg of 6-(4-Tert-Butylphenoxy)Pyridin-3-Amine administered to said subject twice a day. Most preferred is a dose of about 200 mg to about 300 mg of 6-(4-Tert-Butylphenoxy)Pyridin-3-Amine administered to said subject twice a day.

In other embodiments, the dose admininstered twice a day is about 40-60, 60-70, 70-80, 80-90, 90-100, 100-110, 100-140, 140-160, 150-200, 190-210, 200-250, 240-260, 250-300, 250-350, or 300-350 mg of 6-(4-Tert-Butylphenoxy)Pyridin-3-Amine. In other embodiments, the admininstered twice a day is about 40-60, 100-110, 140-160, 190-210, 240-260, 250-300, 250-350, or 300-350 mg of 6-(4-Tert-Butylphenoxy)Pyridin-3-Amine In some embodiments, the pediatric doses can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg twice a day. Thus the dose for patients of age 2 and BW of 15 kg twice a day can be about 5, 10, 15, 20, 25, 30, 35, 40, 45 or 50 mg of 6-(4-Tert-Butylphenoxy)Pyridin-3-Amine, and the dose for patients of age 14 and BW of 65 kg twice a day can be about 20, 40, 60, 90-100 130-150, 170-200, 200-210, or 250-260 mg of 6-(4-Tert-Butylphenoxy)Pyridin-3-, the exact dose is then recalculated for a patient having a lower or higher body weight, or be calcalulated based on body surface area (BSA).

In one embodiment, the 6-(4-Tert-Butylphenoxy)Pyridin-3-Amine monohydrochloride salt is administered twice a day. The period between two administrations per day is usually 8-12 hours.

Thus in one embodiment, the dose administered to said subject of the 6-(4-Tert-Butylphenoxy)Pyridin-3-Amine monohydrochloride salt twice a day is about 30 mg to about 400 mg. In one embodiment, the dose administered to said subject of the 6-(4-Tert-Butylphenoxy)Pyridin-3-Amine monohydrochloride salt twice a day is about 60 mg to about 400 mg. In one embodiment, the dose administered to said subject of the 6-(4-Tert-Butylphenoxy)Pyridin-3-Amine monohydrochloride salt twice a day is about 85 mg to about 400 mg. In one embodiment, the dose administered to said subject of the 6-(4-Tert-Butylphenoxy)Pyridin-3-Amine monohydrochloride salt twice a day is about 125 mg to about 400 mg. In one embodiment, the dose administered to said subject of the 6-(4-Tert-Butylphenoxy)Pyridin-3-Amine monohydrochloride salt twice a day is about 200 mg to about 400 mg. In one embodiment, the dose administered to said subject of the 6-(4-Tert-Butylphenoxy)Pyridin-3-Amine monohydrochloride salt twice a day is about 300 mg to about 400 mg.

In other embodiments, the dose administered to said subject of the 6-(4-Tert-Butylphenoxy)Pyridin-3-Amine monohydrochloride salt twice a day is about 30 mg to about 60 mg, about 30 mg to about 85 mg, about 30 mg to about 125 mg, about 30 mg to about 200 mg, about 30 mg to about 300 mg, or about 30 mg to about 400 mg. In other embodiments, the dose administered to said subject of the 6-(4-Tert-Butylphenoxy)Pyridin-3-Amine monohydrochloride salt twice a day is about 60 mg to about 125 mg, about 60 mg to about 200 mg, about 60 mg to about 300 mg, or about 60 mg to about 400 mg. In other embodiments, the dose administered to said subject of the 6-(4-Tert-Butylphenoxy)Pyridin-3-Amine monohydrochloride salt twice a day is about 50 mg to about 150 mg, about 100 mg to about 200 mg, about 150 mg to about 250 mg, about 200 mg to about 300 mg, about 250 mg to about 350 mg, or about 300 mg to about 400 mg, whererin about 250 mg to about 350 mg is preferred. In a further preferred embodiment the dose administered to said subject of the 6-(4-Tert-Butylphenoxy)Pyridin-3-Amine monohydrochloride salt twice a day is about 30 mg to about 300 mg. In a further preferred embodiment the dose administered to said subject of the 6-(4-Tert-Butylphenoxy)Pyridin-3-Amine monohydrochloride salt twice a day is about 60 mg to about 300 mg. Most preferred is a dose of about 250 mg to about 350 mg of the 6-(4-Tert-Butylphenoxy)Pyridin-3-Amine monohydrochloride salt administered to said subject twice a day.

In some embodiments, the dose administered twice a day is about 30, 60, 65, 70, 75, 80, 85, 90, 95, 100, 110, 120, 125, 130, 140, 150, 160, 170, 175, 180, 190, 200, 210, 220, 225, 230, 240, 250, 260, 270, 275, 280, 290, 300, 310, 320, 325, 330, 340, 350, 360, 370, 380, 390, or 400 mg of the 6-(4-Tert-Butylphenoxy)Pyridin-3-Amine monohydrochloride salt. In other embodiments, the dose is about 30, 60, 75, 85, 100, 125, 150, 175, 200, 225, 250, 275, 300, 325, 350, 375, or 400 mg of the 6-(4-Tert-Butylphenoxy)Pyridin-3-Amine monohydrochloride salt twice a day. In other embodiments, the dose is about 30, 60, 85, 125, 200, or 300 mg of the 6-(4-Tert-Butylphenoxy)Pyridin-3-Amine monohydrochloride salt twice a day.

In some embodiments, the pediatric doses can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg. Thus the dose for patients of age 2 and BW of 15 kg twice a day can be about 10, 20, 30, 40, 50, 60, 70, 80, 90 or 100 mgof the 6-(4-Tert-Butylphenoxy)Pyridin-3-Amine monohydrochloride salt, and the dose for patients of age 14 and BW of 65 kg twice a day can be about 40, 80, 120, 180, 260, 340, 420, or 520 mg of the 6-(4-Tert-Butylphenoxy)Pyridin-3-Amine monohydrochloride salt, the exact dose is then recalculated for a patient having a lower or higher body weight, or be calcalulated based on body surface area (BSA).

6-(4-Tert-Butylphenoxy)Pyridin-3-Amine or one of its salts, solvates, tautomers, or stereoisomers thereof can be administered in the methods of the present invention to a patient who did not receive prior systemic treatment, or was pre-treated with standard of care or any other systemic treatment. Usually, 6-(4-Tert-Butylphenoxy)Pyridin-3-Amine will be given after standard of care for the various cancers. This means that the subject was pretreated with an accepted systemic anticancer treatment such as chemotherapy, endocrine or targeted therapy. However, for ACC and other cancers where no accepted SoC treatment exists, the subjects may be treatment-naive before start of administration of 6-(4-Tert-Butylphenoxy)Pyridin-3-Amine.

In one embodiment, the subject has cancers characterised by activation of the NOTCH signalling pathway as validated by immunohistochemical, molecular and/or biochemical biomarkers.

In the method or use according to the invention, administration of 6-(4-Tert-Butylphenoxy)Pyridin-3-Amine will significantly reduce or stop tumour growth, increase the overall survival (OS), the progression free survival (PFS), the disease-free survival (DFS), and/or the objective response rate (ORR).

In a further aspect, the present invention is directed to 6-(4-Tert-Butylphenoxy)Pyridin-3-Amine or one of its salts, solvates, tautomers, or stereoisomers thereof for use in the treatment of a Notch-dependent cancer in a subject comprising daily administration of about 50 mg to about 700 mg, preferably about 100 mg to about 700 mg, of 6-(4-Tert-Butylphenoxy)Pyridin-3-Amine to said subject. In a further aspect of the present invention, use of 6-(4-Tert-Butylphenoxy)Pyridin-3-Amine or one of its salts, solvates, tautomers, or stereoisomers thereof is provided, in the manufacture of a medicament for treating a Notch-dependent cancer in a subject comprising daily administration of about 50 mg to about 700 mg, preferably about 100 mg to about 700 mg, of 6-(4-Tert-Butylphenoxy)Pyridin-3-Amine to said subject. The following embodiments are embodiments of both aspects of the present invention stated in this paragraph.

In one embodiment, 6-(4-Tert-Butylphenoxy)Pyridin-3-Amine is a hydrochloride salt or a hydrobromide salt, preferably a hydrochloride salt. In a more preferred embodiment, the 6-(4-Tert-Butylphenoxy)Pyridin-3-Amine monohydrochloride salt is used in the present invention.

In one embodiment, the cancer is characterised by activation of the NOTCH signalling pathway optionally characterised by alterations and overactivation of the NOTCH signalling pathway e.g. via genetic alterations such as mutations, translocations, and/or over-expression detectable by appropriate laboratory techniques.

In one embodiment, the subject has a solid tumour such as sarcoma or carcinoma. The solid tumour can be confirmed by histological or cytological methods. In some embodiment the solid tumour is advanced and/or metastatic tumour. In some embodiment the solid tumour is advanced and/or metastatic tumour after standard of care treatment. In certain embodiments, the solid tumour is advanced. The cancer may have spread from where it first started to nearby tissue, lymph nodes, or distant parts of the body. Treatment may be given to help shrink the tumour, slow the growth of cancer cells, or relieve symptoms. And in yet another embodiment, the solid tumour has relapsed or progressed upon or is refractory to standard therapy. In another embodiment, the subject has histologically or cytologically confirmed solid tumours that are surgically unresectable, locally advanced, or metastatic which have progressed on at least one line of systemic therapy.

In one embodiment the subject has a cancer selected from the group consisting of adenoid cystic carcinoma (ACC), breast cancer, prostate cancer, osteosarcoma, malignant glomus tumour, colorectal cancer and hepatocellular carcinoma, preferably selected from the group consisting of adenoid cystic carcinoma (ACC), Triple negative breast cancer (TNBC), ER+ breast cancer, ER− breast cancer, HER2+ breast cancer, HER2− breast cancer, prostate cancer, osteosarcoma, malignant glomus tumour, colorectal cancer in patients resistant to oxaliplatin or irinotecan-based therapy and hepatocellular carcinoma.

In a preferred embodiment the subject has a cancer selected from the group consisting of adenoid cystic carcinoma (ACC), breast cancer, colorectal cancer and prostate cancer. Breast cancer as referred herein comprises hormone receptor positive or negative breast cancers, such as Estrogen receptor positive (ER+), Estrogen receptor negative (ER−), Progesteron receptor positive (PR+), Progesteron receptor negative (PR−), human epidermal growth factor receptor 2 positive (HER2+) and human epidermal growth factor receptor 2 negative (HER2−) breast cancers. The breast cancer can be a triple negative breast cancer (TNBC), where the cancer does not have receptors for either HER2 or the hormones estrogen and progesterone. In some embodiments the breast cancer is selected from the group consisting of triple negative breast cancer, ER- breast cancer, and HER2+ breast cancer. Colorectal cancer as referred herein comprises colorectal cancer wherein the subject is not resistant to oxaliplatin or irinotecan-based therapy and colorectal cancer wherein the subject is resistant to oxaliplatin or irinotecan-based therapy. In a preferred embodiment the colorectal cancer is colorectal cancer wherein the subject is resistant to oxaliplatin or irinotecan-based therapy.

In a particular embodiment, the subject has adenoid cystic carcinoma (ACC). In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating adenoid cystic carcinoma in a subject is provided, comprising administration of 6-(4-Tert-Butylphenoxy)Pyridin-3-Amine or one of its salts, solvates, tautomers, or stereoisomers thereof, to said subject, preferably to a subject who did not receive prior systemic treatment, in particular did not receive other anti-cancer drugs before treatment with 6-(4-Tert-Butylphenoxy)Pyridin-3-Amine.

In a further particular embodiment, the subject has breast cancer, preferably breast cancer selected from the group consisting of triple negative breast cancer, ER− breast cancer, and HER2+ breast cancer. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating breast cancer, preferably breast cancer selected from the group consisting of triple negative breast cancer, ER− breast cancer, and HER2+ breast cancer, in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In a further particular embodiment, the subject has colorectal cancer, preferably colorectal cancer wherein the subject is resistant to oxaliplatin or irinotecan-based therapy. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating colorectal cancer, preferably colorectal cancer wherein the subject is resistant to oxaliplatin or irinotecan-based therapy. in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In a further particular embodiment, the subject has prostate cancer. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating prostate cancer in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In one embodiment the subject has hepatocellular carcinoma. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating hepatocellular carcinoma in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment

In one embodiment the subject has osteosarcoma. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating osteosarcoma in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In one embodiment the subject has a malignant glomus tumour. In one embodiment the subject did not receive prior systemic treatment, in particular did not receive other anti-cancer drugs before treatment with 6-(4-Tert-Butylphenoxy)Pyridin-3-Amine. In one embodiment the subject was pre-treated with standard of care or any other systemic treatment. In one embodiment, a method of treating malignant glomus tumour in a subject is provided, comprising administration of 6-(4-Tert-Butylphenoxy)Pyridin-3-Amine or one of its salts, solvates, tautomers, or stereoisomers thereof, to said subject, preferably to a subject who was pre-treated with standard of care or any other systemic treatment.

In another embodiment, the subject has sarcoma such as osteosarcoma, liposarcoma, rhabdomyosarcoma, or fibrosarcoma. In other embodiments, the subject has gastric cancer, cholangiocellular carcinoma, ovarian cancer, cervical cancer, prostate cancer, non-small cell lung cancer (NSCLC) and especially lung adenocarcinoma which is subgroup of NSCLC, melanoma, or glioblastoma multiforme.

In one embodiment, the subject has osteosarcoma, liposarcoma, rhabdomyosarcoma, or fibrosarcoma, gastric cancer, cholangiocellular carcinoma, ovarian cancer, cervical cancer, prostate cancer, non-small cell lung cancer, lung adenocarcinoma, melanoma, or glioblastoma multiforme.

In another embodiment, the subject has a haematological malignancy. Haematological malignancies can be confirmed by histological or cytological methods. In certain embodiments, the haematological malignancies are advanced. In yet another embodiment, the haematological malignancy has relapsed or progressed upon or is refractory to standard therapy.

In one embodiment the cancer is selected from the group consisting of acute lymphoblastic leukemia, T-cell acute lymphoblastic leukemia (T-ALL) or T-cell lymphoblastic lymphoma (T-LBL), acute myeloid leukemia, chronic lymphocytic leukemia, chronic myelocytic leukemia, Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma.

Thus in one embodiment, the subject has acute lymphoblastic leukemia, T-cell acute lymphoblastic leukemia (T-ALL) or T-cell lymphoblastic lymphoma (T-LBL), acute myeloid leukemia, chronic lymphocytic leukemia, chronic myelocytic leukemia, Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma. In one embodiment, the subject has acute lymphoblastic leukemia, T-cell acute lymphoblastic leukemia (T-ALL) or T-cell lymphoblastic lymphoma (T-LBL), acute myeloid leukemia, chronic lymphocytic leukemia, or chronic myelocytic leukemia of any cell lineage. In another embodiment, the subject has Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma. Thus in a further particular embodiment, the subject has T-cell acute lymphoblastic leukemia (T-ALL).

The multiple myeloma can be relapsed/refractory to at least two lines of therapy, including both a proteasome inhibitor and an immunomodulatory agent. The Hodgkin lymphoma can be relapsed/refractory to at least two lines of treatment including standard of care such as for instance brentuximab vedotin. The B-cell NHL can be relapsed/refractory upon at least one line of standard treatment such as chemo-immunotherapy. The T-cell NHL can be relapsed/refractory upon at least one line of standard treatment such as chemotherapy. The subject with multiple myeloma can have measurable disease (serum M-protein≥10 g/L or urine M-protein≥200 mg/24 hours or abnormal free light chain (FLC) ratio with involved FLC>100 mg/L) or proven plasmacytoma by biopsy). T-cell acute lymphoblastic leukaemia (T-ALL) or T-cell lymphoblastic lymphoma (T-LBL) can be relapsed or refractory (r/r). Refractory patients in this regard are defined as T-ALL/T-LBL patients with ≥5% bone marrow blasts, and/or concomitant extramedullary involvement, who have not achieved a CR after standard induction/consolidation therapy attempt. Relapsed patients are defined as T-ALL/T-LBL patients who have recurrent disease, i.e. ≥5% bone marrow blasts and/or concomitant extramedullary relapse, after having achieved a prior CR.

In one embodiment, the method comprises daily administration of about 100 mg to about 700 mg of 6-(4-Tert-Butylphenoxy)Pyridin-3-Amine. In one embodiment, the method comprises daily administration of about 150 mg to about 700 mg of 6-(4-Tert-Butylphenoxy)Pyridin-3-Amine. In one embodiment, the method comprises daily administration of about 200 mg to about 700 mg of 6-(4-Tert-Butylphenoxy)Pyridin-3-Amine. In one embodiment, the method comprises daily administration of about 350 mg to about 700 mg of 6-(4-Tert-Butylphenoxy)Pyridin-3-Amine. In one embodiment, the method comprises daily administration of about 500 mg to about 700 mg of 6-(4-Tert-Butylphenoxy)Pyridin-3-Amine . In other embodiments, the daily dose administered to said subject of 6-(4-Tert-Butylphenoxy)Pyridin-3-Amine is about 50 mg to about 100 mg, about 50 mg to about 150 mg, about 50 mg to about 200 mg, about 50 mg to about 350 mg, about 50 mg to about 500 mg, or about 50 mg to about 700 mg. In other embodiments, the daily dose administered to said subject of 6-(4-Tert-Butylphenoxy)Pyridin-3-Amine is about 100 mg to about 200 mg, about 100 mg to about 350 mg, about 100 mg to about 500 mg, or about 100 mg to about 700 mg. In other embodiments, the daily dose administered to said subject of 6-(4-Tert-Butylphenoxy)Pyridin-3-Amine is about 100 mg to about 250 mg, about 200 mg to about 350 mg, about 250 mg to about 450 mg, about 350 mg to about 550 mg, about 400 mg to about 650 mg, or about 500 mg to about 700 mg. In a preferred embodiment the daily dose administered to said subject of 6-(4-Tert-Butylphenoxy)Pyridin-3-Amine is about 50 mg to about 500 mg. In a more preferred embodiment the daily dose administered to said subject of 6-(4-Tert-Butylphenoxy)Pyridin-3-Amine is about 100 mg to about 550 mg. In a further more preferred embodiment the daily dose administered to said subject of 6-(4-Tert-Butylphenoxy)Pyridin-3-Amine is about 100 mg to about 900 mg or about 100 mg to about 1100 mg. In an even more preferred embodiment the daily dose administered to said subject of 6-(4-Tert-Butylphenoxy)Pyridin-3-Amine is about 300 mg to about 700 mg. In a further even more preferred embodiment the daily dose administered to said subject of 6-(4-Tert-Butylphenoxy)Pyridin-3-Amine is about 400 mg to about 700 mg. In a further even more preferred embodiment the daily dose administered to said subject of 6-(4-Tert-Butylphenoxy)Pyridin-3-Amine is about 500 mg to about 900 mg or about 500 mg to about 1100 mg. Most preferred is a daily dose of about 400 mg to about 650 mg of 6-(4-Tert-Butylphenoxy)Pyridin-3-Amine, in particular a daily dose of about 500 mg of 6-(4-Tert-Butylphenoxy)Pyridin-3-Amine, administered to said subject. More particular a daily dose of about 800 mg of 6-(4-Tert-Butylphenoxy)Pyridin-3-Amine is administered to said subject. Even more particular a daily dose of about 1000 mg of 6-(4-Tert-Butylphenoxy)Pyridin-3-Amine is administered to said subject.

In some embodiments, the daily dose is about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of 6-(4-Tert-Butylphenoxy)Pyridin-3-Amine per day. In other embodiments, the daily dose is about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, or 1070-1100 mg. In other embodiments, the daily dose is about 100-110, 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, or 1040-1070 mg. In other embodiments, the daily dose is about 100-110, 120-140, 140-160, 170-180, 200-230, 250-270, 290-320, 340-360, 380-400, 440-460, 470-490, 500-550, 550-570, 590-620, 640-660, or 690-710 mg of 6-(4-Tert-Butylphenoxy)Pyridin-3-Amine. In other embodiments, the daily dose is about 45-55, 100-110, 140-160, 200-230, 340-360, or 500-550, or 690-710 or about 45-55, 100-110, 140-160, 200-230, 340-360, or 500-550 mg of 6-(4-Tert-Butylphenoxy)Pyridin-3-Amine. In other embodiments, the daily dose is about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg. In other embodiments, the daily dose is about 100, 120, 150, 170, 200, 250, 300, 350, 400, 450, 500, 550, 600, 650, 700, 750, or 800 mg. In other embodiments, the daily dose is about 120, 150, 170, 200, 250, 300, 350, 400, 450, 500, 550, 600, 650, 700, 750, or 800 mg. In other embodiments, the daily dose is about 60, 120, 170, 250, 400, or 600 mg.

In some embodiments, the dosing can also be weight-based and especially for children and adolescents. The dose can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg. The dose can be about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of 6-(4-Tert-Butylphenoxy)Pyridin-3-Amine. The dose can be about 100-110, 120-140, 140-160, 170-180, 200-230, 250-270, 290-320, 340-360, 380-400, 440-460, 470-490, 500-550, 550-570, 590-620, 640-660, or 690-710 mg of 6-(4-Tert-Butylphenoxy)Pyridin-3-Amine per 70 kg patient, the exact dose is then recalculated for a patient having a lower or higher body weight than 70 kg. In another embodiment, the dose can be about 45-55, 100-110, 140-160, 200-230, 340-360, 500-550, or 690-710 or about 45-55, 100-110, 140-160, 200-230, 340-360, or 500-550 mg of 6-(4-Tert-Butylphenoxy)Pyridin-3-Amine per 70 kg patient. In another embodiment, the dose can be about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg per 70 kg patient. In other embodiments, the daily dose is about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, or 1070-1100 mg per 70 kg patient. In other embodiments, the daily dose is about 100-110, 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, or 1040-1070 mg per 70 kg patient. In other embodiments, the daily dose is about 100, 120, 150, 170, 200, 250, 300, 350, 400, 450, 500, 550, 600, 650, 700, 750, or 800 mg per 70 kg patient. In another embodiment, the dose can be about 120, 150, 170, 200, 250, 300, 350, 400, 450, 500, 550, 600, 650, 700, 750, or 800 mg. In another embodiment, the dose can be about about 60, 120, 170, 250, 400, or 600 mg. The exact dose is then recalculated for a patient having a lower or higher body weight than 70 kg, or be calcalulated based on body surface area (BSA).

In one embodiment, the method comprises daily administration of about 120 mg to about 800 mg of the 6-(4-Tert-Butylphenoxy)Pyridin-3-Amine monohydrochloride salt. In one embodiment, the method comprises daily administration of about 170 mg to about 800 mg of the 6-(4-Tert-Butylphenoxy)Pyridin-3-Amine monohydrochloride salt. In one embodiment, the method comprises daily administration of about 250 mg to about 800 mg of the 6-(4-Tert-Butylphenoxy)Pyridin-3-Amine monohydrochloride salt. In one embodiment, the method comprises daily administration of about 400 mg to about 800 mg of the 6-(4-Tert-Butylphenoxy)Pyridin-3-Amine monohydrochloride salt. In one embodiment, the method comprises daily administration of about 600 mg to about 800 mg of the 6-(4-Tert-Butylphenoxy)Pyridin-3-Amine monohydrochloride salt. In other embodiments, the daily dose administered to said subject of the 6-(4-Tert-Butylphenoxy)Pyridin-3-Amine monohydrochloride salt is about 60 mg to about 120 mg, about 60 mg to about 170 mg, about 60 mg to about 250 mg, about 60 mg to about 400 mg, about 60 mg to about 600 mg, or about 60 mg to about 800 mg. In other embodiments, the daily dose administered to said subject of the 6-(4-Tert-Butylphenoxy)Pyridin-3-Amine monohydrochloride salt is about 120 mg to about 250 mg, about 120 mg to about 400 mg, about 120 mg to about 600 mg, or about 120 mg to about 800 mg. In other embodiments, the daily dose administered to said subject of the 6-(4-Tert-Butylphenoxy)Pyridin-3-Amine monohydrochloride salt is about 100 mg to about 300mg, about 200 mg to about 400 mg, about 300 mg to about 500 mg, about 400 mg to about 600 mg, about 500 mg to about 700 mg, or about 600 mg to about 800 mg. In a preferred embodiment the daily dose administered to said subject of the 6-(4-Tert-Butylphenoxy)Pyridin-3-Amine monohydrochloride salt is about 60 mg to about 600 mg. In a more preferred embodiment the daily dose administered to said subject of the 6-(4-Tert-Butylphenoxy)Pyridin-3-Amine monohydrochloride salt is about 120 mg to about 600 mg. In a further more preferred embodiment the daily dose administered to said subject of the 6-(4-Tert-Butylphenoxy)Pyridin-3-Amine monohydrochloride salt is about 120 mg to about 1000 mg or about 120 mg to about 1200 mg. In an even more preferred embodiment the daily dose administered to said subject of the 6-(4-Tert-Butylphenoxy)Pyridin-3-Amine monohydrochloride salt is about 120 mg to about 800 mg. In a further even more preferred embodiment the daily dose administered to said subject of the 6-(4-Tert-Butylphenoxy)Pyridin-3-Amine monohydrochloride salt is about 600 mg to about 1000 mg or about 600 mg to about 1200 mg. Most preferred is a daily dose of about 500 mg to about 700 mg of the 6-(4-Tert-Butylphenoxy)Pyridin-3-Amine monohydrochloride salt, in particular about 600 mg of the 6-(4-Tert-Butylphenoxy)Pyridin-3-Amine monohydrochloride salt, administered to said subject. More particular a daily dose of about 900 mg of the 6-(4-Tert-Butylphenoxy)Pyridin-3-Amine monohydrochloride salt is administered to said subject. Even more particular a daily dose of about 1150 mg of the 6-(4-Tert-Butylphenoxy)Pyridin-3-Amine monohydrochloride salt is administered to said subject.

In some embodiments, the daily dose is about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg of the 6-(4-Tert-Butylphenoxy)Pyridin-3-Amine monohydrochloride salt. In other embodiments, the daily dose is about 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of the 6-(4-Tert-Butylphenoxy)Pyridin-3-Amine monohydrochloride salt. In other embodiments, the daily dose is about 120-140, 140-160, 170-180, 200-230, 250-270, 290-320, 340-360, 380-400, 440-460, 470-490, 500-550, 550-570, 590-620, 640-660, or 690-710 mg of the 6-(4-Tert-Butylphenoxy)Pyridin-3-Amine monohydrochloride salt. In other embodiments, the daily dose is about120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, 1070-1100, 1100-1130, 1130-1160, or 1160-1200 mg of the 6-(4-Tert-Butylphenoxy)Pyridin-3-Amine monohydrochloride salt. In other embodiments, the daily dose is about about 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, 1040-1070, 1100-1130, or 1160-1200 mg of the 6-(4-Tert-Butylphenoxy)Pyridin-3-Amine monohydrochloride salt. In other embodiments, the daily dose is about 120, 150, 170, 200, 250, 300, 350, 400, 450, 500, 550, 600, 650, 700, 750, or 800 mg of the 6-(4-Tert-Butylphenoxy)Pyridin-3-Amine monohydrochloride salt. In other embodiments, the daily dose is about 60, 120, 170, 250, 400, or 600 mg of the 6-(4-Tert-Butylphenoxy)Pyridin-3-Amine monohydrochloride salt.

In some embodiments, the dosing can also be weight-based and especially for children and adolescents. The dose can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg. The dose can be 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780 or 800 mg of the 6-(4-Tert-Butylphenoxy)Pyridin-3-Amine monohydrochloride salt per 70 kg patient, the exact dose is then recalculated for a patient having a lower or higher body weight than 70 kg. In another embodiment, the dose can be 60, 120, 170, 250, 400, 600, 700, or 800 mg of the 6-(4-Tert-Butylphenoxy)Pyridin-3-Amine monohydrochloride salt per 70 kg patient, the exact dose is then recalculated for a patient having a lower or higher body weight than 70 kg, or be calcalulated based on body surface area (BSA). In other embodiments, the daily dose is about 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of the 6-(4-Tert-Butylphenoxy)Pyridin-3-Amine monohydrochloride salt per 70 kg patient. In other embodiments, the daily dose is about 120-140, 140-160, 170-180, 200-230, 250-270, 290-320, 340-360, 380-400, 440-460, 470-490, 500-550, 550-570, 590-620, 640-660, or 690-710 mg of the 6-(4-Tert-Butylphenoxy)Pyridin-3-Amine monohydrochloride salt per 70 kg patient. In other embodiments, the daily dose is about120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, 1070-1100, 1100-1130, 1130-1160, or 1160-1200 mg of the 6-(4-Tert-Butylphenoxy)Pyridin-3-Amine monohydrochloride salt per 70 kg patient. In other embodiments, the daily dose is about about 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, 1040-1070, 1100-1130, or 1160-1200 mg of the 6-(4-Tert-Butylphenoxy)Pyridin-3-Amine monohydrochloride salt per 70 kg patient.

In one embodiment, 6-(4-Tert-Butylphenoxy)Pyridin-3-Amine is administered twice a day. The period between two administrations per day is usually 8-12 hours.

Thus in one embodiment, the dose administered to said subject of 6-(4-Tert-Butylphenoxy)Pyridin-3-Amine twice a day is about 12.5 mg to about 350 mg. In one embodiment, the dose administered to said subject of 6-(4-Tert-Butylphenoxy)Pyridin-3-Amine twice a day is about 25 mg to about 350 mg. In one embodiment, the dose administered to said subject of 6-(4-Tert-Butylphenoxy)Pyridin-3-Amine twice a day is about 75 mg to about 350 mg. In one embodiment, the dose administered to said subject of 6-(4-Tert-Butylphenoxy)Pyridin-3-Amine twice a day is about 100 mg to about 350 mg. In one embodiment, the dose administered to said subject of 6-(4-Tert-Butylphenoxy)Pyridin-3-Amine twice a day is about 150 mg to about 350 mg. In one embodiment, the dose administered to said subject of 6-(4-Tert-Butylphenoxy)Pyridin-3-Amine twice a day is about 250 mg to about 350 mg.

In other embodiments, the dose administered to said subject of 6-(4-Tert-Butylphenoxy)Pyridin-3-Amine twice a day is about 12.5 mg to about 25 mg, about 12.5 mg to about 50 mg, about 12.5 mg to about 75 mg, about 12.5 mg to about 100 mg, about 12.5 mg to about 175 mg, about 12.5 mg to about 250 mg, or about 12.5 mg to about 350 mg. In other embodiments, the dose administered to said subject of 6-(4-Tert-Butylphenoxy)Pyridin-3-Amine twice a day is about 25 mg to about 50 mg, about 25 mg to about 75 mg, about 25 mg to about 100 mg, about 25 mg to about 150 mg, or about 25 mg to about 200 mg. In other embodiments, the dose administered to said subject of 6-(4-Tert-Butylphenoxy)Pyridin-3-Amine twice a day is about 50 mg to about 150 mg, about 100 mg to about 200 mg, about 100 mg to about 250 mg, about 200 mg to about 250 mg, about 200 mg to about 300 mg, or about 250 mg to about 350 mg, wherein 200 mg to about 300 mg is preferred. In a further preferred embodiment the dose administered to said subject of 6-(4-Tert-Butylphenoxy)Pyridin-3-Amine twice a day is about 12.5 mg to about 250 mg. In a further preferred embodiment the dose administered to said subject of 6-(4-Tert-Butylphenoxy)Pyridin-3-Amine twice a day is about 25 mg to about 250 mg. In a further preferred embodiment the dose administered to said subject of 6-(4-Tert-Butylphenoxy)Pyridin-3-Amine twice a day is about 150 mg to about 350 mg. Particular preferred is a dose of about 200 mg to about 350 mg of 6-(4-Tert-Butylphenoxy)Pyridin-3-Amine administered to said subject twice a day. Most preferred is a dose of about 200 mg to about 300 mg of 6-(4-Tert-Butylphenoxy)Pyridin-3-Amine administered to said subject twice a day.

In other embodiments, the dose admininstered twice a day is about 40-60, 60-70, 70-80, 80-90, 90-100, 100-110, 100-140, 140-160, 150-200, 190-210, 200-250, 240-260, 250-300, 250-350, or 300-350 mg of 6-(4-Tert-Butylphenoxy)Pyridin-3-Amine. In other embodiments, the admininstered twice a day is about 40-60, 100-110, 140-160, 190-210, 240-260, 250-300, 250-350, or 300-350 mg of 6-(4-Tert-Butylphenoxy)Pyridin-3-Amine In some embodiments, the pediatric doses can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg twice a day. Thus the dose for patients of age 2 and BW of 15 kg twice a day can be about 5, 10, 15, 20, 25, 30, 35, 40, 45 or 50 mg of 6-(4-Tert-Butylphenoxy)Pyridin-3-Amine, and the dose for patients of age 14 and BW of 65 kg twice a day can be about 20, 40, 60, 90-100 130-150, 170-200, 200-210, or 250-260 mg of 6-(4-Tert-Butylphenoxy)Pyridin-3-, the exact dose is then recalculated for a patient having a lower or higher body weight, or be calcalulated based on body surface area (BSA).

In one embodiment, the 6-(4-Tert-Butylphenoxy)Pyridin-3-Amine monohydrochloride salt is administered twice a day. The period between two administrations per day is usually 8-12 hours.

Thus in one embodiment, the dose administered to said subject of the 6-(4-Tert-Butylphenoxy)Pyridin-3-Amine monohydrochloride salt twice a day is about 30 mg to about 400 mg. In one embodiment, the dose administered to said subject of the 6-(4-Tert-Butylphenoxy)Pyridin-3-Amine monohydrochloride salt twice a day is about 60 mg to about 400 mg. In one embodiment, the dose administered to said subject of the 6-(4-Tert-Butylphenoxy)Pyridin-3-Amine monohydrochloride salt twice a day is about 85 mg to about 400 mg. In one embodiment, the dose administered to said subject of the 6-(4-Tert-Butylphenoxy)Pyridin-3-Amine monohydrochloride salt twice a day is about 125 mg to about 400 mg. In one embodiment, the dose administered to said subject of the 6-(4-Tert-Butylphenoxy)Pyridin-3-Amine monohydrochloride salt twice a day is about 200 mg to about 400 mg. In one embodiment, the dose administered to said subject of the 6-(4-Tert-Butylphenoxy)Pyridin-3-Amine monohydrochloride salt twice a day is about 300 mg to about 400 mg.

In other embodiments, the dose administered to said subject of the 6-(4-Tert-Butylphenoxy)Pyridin-3-Amine monohydrochloride salt twice a day is about 30 mg to about 60 mg, about 30 mg to about 85 mg, about 30 mg to about 125 mg, about 30 mg to about 200 mg, about 30 mg to about 300 mg, or about 30 mg to about 400 mg. In other embodiments, the dose administered to said subject of the 6-(4-Tert-Butylphenoxy)Pyridin-3-Amine monohydrochloride salt twice a day is about 60 mg to about 125 mg, about 60 mg to about 200 mg, about 60 mg to about 300 mg, or about 60 mg to about 400 mg. In other embodiments, the dose administered to said subject of the 6-(4-Tert-Butylphenoxy)Pyridin-3-Amine monohydrochloride salt twice a day is about 50 mg to about 150 mg, about 100 mg to about 200 mg, about 150 mg to about 250 mg, about 200 mg to about 300 mg, about 250 mg to about 350 mg, or about 300 mg to about 400 mg, whererin about 250 mg to about 350 mg is preferred. In a further preferred embodiment the dose administered to said subject of the 6-(4-Tert-Butylphenoxy)Pyridin-3-Amine monohydrochloride salt twice a day is about 30 mg to about 300 mg. In a further preferred embodiment the dose administered to said subject of the 6-(4-Tert-Butylphenoxy)Pyridin-3-Amine monohydrochloride salt twice a day is about 60 mg to about 300 mg. Most preferred is a dose of about 250 mg to about 350 mg of the 6-(4-Tert-Butylphenoxy)Pyridin-3-Amine monohydrochloride salt administered to said subject twice a day.

In some embodiments, the dose administered twice a day is about 30, 60, 65, 70, 75, 80, 85, 90, 95, 100, 110, 120, 125, 130, 140, 150, 160, 170, 175, 180, 190, 200, 210, 220, 225, 230, 240, 250, 260, 270, 275, 280, 290, 300, 310, 320, 325, 330, 340, 350, 360, 370, 380, 390, or 400 mg of the 6-(4-Tert-Butylphenoxy)Pyridin-3-Amine monohydrochloride salt. In other embodiments, the dose is about 30, 60, 75, 85, 100, 125, 150, 175, 200, 225, 250, 275, 300, 325, 350, 375, or 400 mg of the 6-(4-Tert-Butylphenoxy)Pyridin-3-Amine monohydrochloride salt twice a day. In other embodiments, the dose is about 30, 60, 85, 125, 200, or 300 mg of the 6-(4-Tert-Butylphenoxy)Pyridin-3-Amine monohydrochloride salt twice a day.

In some embodiments, the pediatric doses can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg. Thus the dose for patients of age 2 and BW of 15 kg twice a day can be about 10, 20, 30, 40, 50, 60, 70, 80, 90 or 100 mgof the 6-(4-Tert-Butylphenoxy)Pyridin-3-Amine monohydrochloride salt, and the dose for patients of age 14 and BW of 65 kg twice a day can be about 40, 80, 120, 180, 260, 340, 420, or 520 mg of the 6-(4-Tert-Butylphenoxy)Pyridin-3-Amine monohydrochloride salt, the exact dose is then recalculated for a patient having a lower or higher body weight, or be calcalulated based on body surface area (BSA).

6-(4-Tert-Butylphenoxy)Pyridin-3-Amine or one of its salts, solvates, tautomers, or stereoisomers thereof can be administered in the methods of the present invention to a patient who did not receive prior systemic treatment, or was pre-treated with standard of care or any other systemic treatment. Usually, 6-(4-Tert-Butylphenoxy)Pyridin-3-Amine will be given after standard of care for the various cancers. This means that the subject was pretreated with an accepted systemic anticancer treatment such as chemotherapy, endocrine or targeted therapy. However, for ACC and other cancers where no accepted SoC treatment exists, the subjects may be treatment-naïve before start of administration of 6-(4-Tert-Butylphenoxy)Pyridin-3-Amine.

In one embodiment, the subject has cancers characterised by activation of the NOTCH signalling pathway as validated by immunohistochemical, molecular and/or biochemical biomarkers.

In the method or use according to the invention, administration of 6-(4-Tert-Butylphenoxy)Pyridin-3-Amine will significantly reduce or stop tumour growth, increase the overall survival (OS), the progression free survival (PFS), the disease-free survival (DFS), and/or the objective response rate (ORR).

In a further aspect, the present invention provides a pharmaceutical composition in unit dose comprising 6-(4-Tert-Butylphenoxy)Pyridin-3-Amine or one of its salts, solvates, tautomers, or stereoisomers thereof, wherein the pharmaceutical composition comprises about 50 mg to about 700 mg, preferably about 100 mg to about 700 mg, of 6-(4-Tert-Butylphenoxy)Pyridin-3-Amine, and a pharmaceutically acceptable carrier. In a further aspect, the present invention provides a pharmaceutical composition comprising 6-(4-Tert-Butylphenoxy)Pyridin-3-Amine or one of its salts, solvates, tautomers, or stereoisomers thereof, wherein the pharmaceutical composition comprises about 50 mg to about 700 mg, preferably about 100 mg to about 700 mg, of 6-(4-Tert-Butylphenoxy)Pyridin-3-Amine, for use in the treatment of a Notch-dependent cancer in a subject, and a pharmaceutically acceptable carrier. The following embodiments are embodiments of both aspects of the present invention stated in this paragraph.

In one embodiment, 6-(4-Tert-Butylphenoxy)Pyridin-3-Amine is a hydrochloride salt or a hydrobromide salt, preferably a hydrochloride salt. In a more preferred embodiment, the 6-(4-Tert-Butylphenoxy)Pyridin-3-Amine monohydrochloride salt is used in the present invention. In one embodiment, the pharmaceutical composition comprises a unit dose of about 100 mg to about 700 mg of 6-(4-Tert-Butylphenoxy)Pyridin-3-Amine. In one embodiment, the pharmaceutical composition comprises a unit dose of about 150 mg to about 700 mg of 6-(4-Tert-Butylphenoxy)Pyridin-3-Amine. In one embodiment, the pharmaceutical composition comprises a unit dose of about 200 mg to about 700 mg of 6-(4-Tert-Butylphenoxy)Pyridin-3-Amine. In one embodiment, the pharmaceutical composition comprises a unit dose of about 350 mg to about 700 mg of 6-(4-Tert-Butylphenoxy)Pyridin-3-Amine. In one embodiment, the pharmaceutical composition comprises a unit dose of about 500 mg to about 700 mg of 6-(4-Tert-Butylphenoxy)Pyridin-3-Amine. In other embodiments, the pharmaceutical composition comprises a unit dose of about 50 mg to about 100 mg, about 50 mg to about 150 mg, about 50 mg to about 200 mg, about 50 mg to about 350 mg, about 50 mg to about 550 mg, or about 50 mg to about 700 mg of 6-(4-Tert-Butylphenoxy)Pyridin-3-Amine. In other embodiments, the pharmaceutical composition comprises a unit dose of 6-(4-Tert-Butylphenoxy)Pyridin-3-Amine of about 100 mg to about 200 mg, about 100 mg to about 350 mg, about 100 mg to about 550 mg, or about 100 mg to about 700 mg. In other embodiments, the pharmaceutical composition comprises a unit dose of 6-(4-Tert-Butylphenoxy)Pyridin-3-Amine of about 100 mg to about 250mg, about 200 mg to about 350 mg, about 250 mg to about 450 mg, about 350 mg to about 550 mg, about 400 mg to about 650 mg, or about 500 mg to about 700 mg. In a preferred embodiment the pharmaceutical composition comprises a unit dose of about 50 mg to about 500 mg of 6-(4-Tert-Butylphenoxy)Pyridin-3-Amine. In a further preferred embodiment the pharmaceutical composition comprises a unit dose of is about 50 mg to about 550 mg of 6-(4-Tert-Butylphenoxy)Pyridin-3-Amine. In a more preferred embodiment the pharmaceutical composition comprises a unit dose of 6-(4-Tert-Butylphenoxy)Pyridin-3-Amine of about 100 mg to about 550 mg of 6-(4-Tert-Butylphenoxy)Pyridin-3-Amine. In a further more preferred embodiment the pharmaceutical composition comprises a unit dose of 6-(4-Tert-Butylphenoxy)Pyridin-3-Amine of about 100 mg to about 900 mg or about 100 mg to about 1100 mg. In an even more preferred embodiment the pharmaceutical composition comprises a unit dose of 6-(4-Tert-Butylphenoxy)Pyridin-3-Amine of about 300 mg to about 700 mg. In a further even more preferred embodiment the pharmaceutical composition comprises a unit dose of 6-(4-Tert-Butylphenoxy)Pyridin-3-Amine of about 400 mg to about 700 mg. In a further even more preferred embodiment the pharmaceutical composition comprises a unit dose of 6-(4-Tert-Butylphenoxy)Pyridin-3-Amine of about 500 mg to about 900 mg or about 500 mg to about 1100 mg. Most preferred is a pharmaceutical composition comprising a unit dose of about 400 mg to about 650 mg of 6-(4-Tert-Butylphenoxy)Pyridin-3-Amine, in particular a pharmaceutical composition comprising a unit dose of about 500 mg of 6-(4-Tert-Butylphenoxy)Pyridin-3-Amine. More particularly preferred is a pharmaceutical composition comprising a unit dose of about 800 mg of 6-(4-Tert-Butylphenoxy)Pyridin-3-Amine. Even more particularly preferred is a pharmaceutical composition comprising a unit dose of about 1000 mg of 6-(4-Tert-Butylphenoxy)Pyridin-3-Amine.

In some embodiments, the pharmaceutical composition comprises a unit dose of about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of 6-(4-Tert-Butylphenoxy)Pyridin-3-Amine per day. In other embodiments the pharmaceutical composition comprises a unit dose of about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, or 1070-1100 mg. In other embodiments, the pharmaceutical composition comprises a unit dose of about 100-110, 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, or 1040-1070 mg. In other embodiments, the pharmaceutical composition comprises a unit dose of about 100-110, 120-140, 140-160, 170-180, 200-230, 250-270, 290-320, 340-360, 380-400, 440-460, 470-490, 500-550, 550-570, 590-620, 640-660, or 690-710 mg of 6-(4-Tert-Butylphenoxy)Pyridin-3-Amine. In other embodiments, the pharmaceutical composition comprises a unit dose of about 45-55, 100-110, 140-160, 200-230, 340-360, or 500-550, or 690-710 or about 45-55, 100-110, 140-160, 200-230, 340-360, or 500-550 mg of 6-(4-Tert-Butylphenoxy)Pyridin-3-Amine. In other embodiments, the pharmaceutical composition comprises a unit dose about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg. In other embodiments, the pharmaceutical composition comprises a unit dose of about 100, 120, 150, 170, 200, 250, 300, 350, 400, 450, 500, 550, 600, 650, 700, 750, or 800 mg. In other embodiments, the daily dose is about 120, 150, 170, 200, 250, 300, 350, 400, 450, 500, 550, 600, 650, 700, 750, or 800 mg. In other embodiments, the pharmaceutical composition comprises a unit dose about 60, 120, 170, 250, 400, or 600 mg.

In some embodiments, the dosing can also be weight-based and especially for children and adolescents. The dose can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg. The dose can be about 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of 6-(4-Tert-Butylphenoxy)Pyridin-3-Amine. The dose can be about 100-110, 120-140, 140-160, 170-180, 200-230, 250-270, 290-320, 340-360, 380-400, 440-460, 470-490, 500-550, 550-570, 590-620, 640-660, or 690-710 mg of 6-(4-Tert-Butylphenoxy)Pyridin-3-Amine per 70 kg patient, the exact dose is then recalculated for a patient having a lower or higher body weight than 70 kg. In another embodiment, the dose can be 45-55, 100-110, 140-160, 200-230, 340-360, 500-550, or 690-710 or about 45-55, 100-110, 140-160, 200-230, 340-360, or 500-550 mg of 6-(4-Tert-Butylphenoxy)Pyridin-3-Amine per 70 kg patient. In another embodiment, the dose can be about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg per 70 kg patient. In other embodiments, the dose can be bout 100-110, 110-120, 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, or 1070-1100 mg per 70 kg patient. In other embodiments, the daily dose can be about 100-110, 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, or 1040-1070 mg per 70 kg patient. In other embodiments, the daily dose can be about 100, 120, 150, 170, 200, 250, 300, 350, 400, 450, 500, 550, 600, 650, 700, 750, or 800 mg per 70 kg patient. In another embodiment, the dose can be about 120, 150, 170, 200, 250, 300, 350, 400, 450, 500, 550, 600, 650, 700, 750, or 800 mg. In another embodiment, the dose can be about about 60, 120, 170, 250, 400, or 600 mg. The exact dose is then recalculated for a patient having a lower or higher body weight than 70 kg, or be calcalulated based on body surface area (BSA).

In embodiments where the pharmaceutical composition is adminestered twic a day, the unit dose can be as follows. In one embodiment, the pharmaceutical composition comprises a unit dose of 6-(4-Tert-Butyl-phenoxy)Pyridin-3-Amine of about 12.5 mg to about 350 mg. In one embodiment, the pharmaceutical composition comprises a unit dose of 6-(4-Tert-Butylphenoxy)Pyridin-3-Amine of about 25 mg to about 350 mg. In one embodiment, the pharmaceutical composition comprises a unit dose of 6-(4-Tert-Butylphenoxy)Pyridin-3-Amine of about 75 mg to about 350 mg. In one embodiment, the pharmaceutical composition comprises a unit dose of 6-(4-Tert-Butylphenoxy)Pyridin-3-Amine of about 100 mg to about 350 mg. In one embodiment, the pharmaceutical composition comprises a unit dose of 6-(4-Tert-Butylphenoxy)Pyridin-3-Amine of about 150 mg to about 350 mg. In one embodiment, the pharmaceutical composition comprises a unit dose of 6-(4-Tert-Butylphenoxy)Pyridin-3-Amine of about 250 mg to about 350 mg.

In other embodiments, the pharmaceutical composition comprises a unit dose of 6-(4-Tert-Butylphenoxy)Pyridin-3-Amine of about 12.5 mg to about 25 mg, about 12.5 mg to about 50 mg, about 12.5 mg to about 75 mg, about 12.5 mg to about 100 mg, about 12.5 mg to about 175 mg, about 12.5 mg to about 250 mg, or about 12.5 mg to about 350 mg. In other embodiments, the pharmaceutical composition comprises a unit dose of 6-(4-Tert-Butylphenoxy)Pyridin-3-Amine of is about 25 mg to about 50 mg, about 25 mg to about 75 mg, about 25 mg to about 100 mg, mg, about 25 mg to about 150 mg, or about 25 mg to about 200 mg. In other embodiments, the pharmaceutical composition comprises a unit dose of 6-(4-Tert-Butylphenoxy)Pyridin-3-Amine of about 50 mg to about 150 mg, about 100 mg to about 200 mg, about 100 mg to about 250 mg, about 200 mg to about 250 mg, about 200 mg to about 300 mg, or about 250 mg to about 350 mg, wherein 200 mg to about 300 mg is preferred. In a further preferred embodiment the pharmaceutical composition comprises a unit dose of 6-(4-Tert-Butylphenoxy)Pyridin-3-Amine of about 12.5 mg to about 250 mg. In a further preferred embodiment the pharmaceutical composition comprises a unit dose of 6-(4-Tert-Butylphenoxy)Pyridin-3-Amine of about 25 mg to about 250 mg. In a further preferred embodiment the pharmaceutical composition comprises a unit dose of 6-(4-Tert-Butylphenoxy)Pyridin-3-Amine of about 150 mg to about 350 mg. Particular preferrably the pharmaceutical composition comprises a unit dose of about 200 mg to about 350 mg of 6-(4-Tert-Butylphenoxy)Pyridin-3-Amine. Most preferrably the pharmaceutical composition comprises a unit dose of 6-(4-Tert-Butylphenoxy)Pyridin-3-Amine of about 200 mg to about 300 mg.

In other embodiments, the pharmaceutical composition comprises a unit dose of 6-(4-Tert-Butylphenoxy)Pyridin-3-Amine of about 40-60, 60-70, 70-80, 80-90, 90-100, 100-110, 100-140, 140-160, 150-200, 190-210, 200-250, 240-260, 250-300, 250-350, or 300-350 mg. In other embodiments, the pharmaceutical composition comprises a unit dose of 6-(4-Tert-Butylphenoxy)Pyridin-3-Amine of about 40-60, 100-110, 140-160, 190-210, 240-260, 250-300, 250-350, or 300-350 mg.

In some embodiments, the pediatric doses can for instance be 1.0-9.0 mg/kg, 1.2-6.0 mg/kg, 1.5-4.0 mg/kg, 2.0-5.0 mg/kg, 3.0-6.0 mg/kg, 4.0-7.0 mg/kg, 6.0-9.0 mg/kg, or 4.0-9.0 mg/kg twice a day. Thus the unit dose for patients of age 2 and BW of 15 kg twice a day can be about 5, 10, 15, 20, 25, 30, 35, 40, 45 or 50 mg of 6-(4-Tert-Butylphenoxy)Pyridin-3-Amine, and the unit dose for patients of age 14 and BW of 65 kg twice a day can be about 20, 40, 60, 90-100 130-150, 170-200, 200-210, or 250-260 mg of 6-(4-Tert-Butylphenoxy)Pyridin-3-Amine, the exact dose is then recalculated for a patient having a lower or higher body weight, or be calcalulated based on body surface area (BSA).

In one embodiment, the pharmaceutical composition comprises a unit dose of about 120 mg to about 800 mg of the 6-(4-Tert-Butylphenoxy)Pyridin-3-Amine monohydrochloride salt. In one embodiment, the pharmaceutical composition comprises a unit dose of about 170 mg to about 800 mg of the 6-(4-Tert-Butylphenoxy)Pyridin-3-Amine monohydrochloride salt. In one embodiment, the pharmaceutical composition comprises a unit dose of about 250 mg to about 800 mg of the 6-(4-Tert-Butylphenoxy)Pyridin-3-Amine monohydrochloride salt. In one embodiment, the pharmaceutical composition comprises a unit dose of about 400 mg to about 800 mg of the 6-(4-Tert-Butylphenoxy)Pyridin-3-Amine monohydrochloride salt. In one embodiment, the pharmaceutical composition comprises a unit dose of about 600 mg to about 800 mg of the 6-(4-Tert-Butylphenoxy)Pyridin-3-Amine monohydrochloride salt. In other embodiments, the pharmaceutical composition comprises a unit dose of about 60 mg to about 120 mg, about 60 mg to about 170 mg, about 60 mg to about 250 mg, about 60 mg to about 400 mg, about 60 mg to about 600 mg, or about 60 mg to about 800 mg of the 6-(4-Tert-Butylphenoxy)Pyridin-3-Amine monohydrochloride salt. In other embodiments, the pharmaceutical composition comprises a unit dose of about 120 mg to about 250 mg, about 120 mg to about 400 mg, about 120 mg to about 600 mg, or about 120 mg to about 800 mg of the 6-(4-Tert-Butylphenoxy)Pyridin-3-Amine monohydrochloride salt. In other embodiments, the pharmaceutical composition comprises a unit dose of about 100 mg to about 300mg, about 200 mg to about 400 mg, about 300 mg to about 500 mg, about 400 mg to about 600 mg, about 500 mg to about 700 mg, or about 600 mg to about 800 mg of the 6-(4-Tert-Butylphenoxy)Pyridin-3-Amine monohydrochloride salt, wherein 500 mg to about 700 mg is preferred . In a further preferred embodiment the pharmaceutical composition comprises a unit dose of about 60 mg to about 600 mg of the 6-(4-Tert-Butylphenoxy)Pyridin-3-Amine monohydrochloride salt. In a more preferred embodiment the pharmaceutical composition comprises a unit dose of about 120 mg to about 600 mg of the 6-(4-Tert-Butylphenoxy)Pyridin-3-Amine monohydrochloride salt. In a further more preferred embodiment the pharmaceutical composition comprises a unit dose of about 120 mg to about 1000 mg or about 120 mg to about 1200 mg of the 6-(4-Tert-Butylphenoxy)Pyridin-3-Amine monohydrochloride salt. In an even more preferred embodiment the pharmaceutical composition comprises a unit dose of about 600 mg to about 1000 mg or about 600 mg to about 1200 mg of the 6-(4-Tert-Butylphenoxy)Pyridin-3-Amine monohydrochloride salt. Most preferred is a pharmaceutical composition comprising a unit dose of about 250 mg to about 350 mg of the 6-(4-Tert-Butylphenoxy)Pyridin-3-Amine monohydrochloride salt, in particular a pharmaceutical composition comprising a unit dose of about 500 mg to about 700 mg of the 6-(4-Tert-Butylphenoxy)Pyridin-3-Amine monohydrochloride salt, more particular a pharmaceutical composition comprising a unit dose of about 600 mg of the 6-(4-Tert-Butylphenoxy)Pyridin-3-Amine monohydrochloride salt. Even more particularly preferred is a pharmaceutical composition comprising a unit dose of about 900 mg of the 6-(4-Tert-Butylphenoxy)Pyridin-3-Amine monohydrochloride salt. Most particularly preferred is a pharmaceutical composition comprising a unit dose of about 1150 mg of the 6-(4-Tert-Butylphenoxy)Pyridin-3-Amine monohydrochloride salt.

In one embodiment the composition comprises a unit dose of about 30 mg to about 400 mg of the 6-(4-Tert-Butylphenoxy)Pyridin-3-Amine monohydrochloride salt. In one embodiment, the composition comprises a unit dose of about 60 mg to about 400 mg of the 6-(4-Tert-Butylphenoxy)Pyridin-3-Amine monohydrochloride salt. In one embodiment, the composition comprises a unit dose of about 85 mg to about 400 mg of the 6-(4-Tert-Butylphenoxy)Pyridin-3-Amine monohydrochloride salt. In one embodiment, the composition comprises a unit dose of about 125 mg to about 400 mg of the 6-(4-Tert-Butylphenoxy)Pyridin-3-Amine monohydrochloride salt. In one embodiment, the composition comprises a unit dose of about 200 mg to about 400 mg of the 6-(4-Tert-Butylphenoxy)Pyridin-3-Amine monohydrochloride salt. In one embodiment, the composition comprises a unit dose of about 300 mg to about 400 mg of the 6-(4-Tert-Butylphenoxy)Pyridin-3-Amine monohydrochloride salt.

In other embodiments, the composition comprises a unit dose of about 30 mg to about 60 mg, about 30 mg to about 85 mg, about 30 mg to about 125 mg, about 30 mg to about 200 mg, about 30 mg to about 300 mg, or about 30 mg to about 400 mg of the 6-(4-Tert-Butylphenoxy)Pyridin-3-Amine monohydrochloride salt. In other embodiments, the composition comprises a unit dose of about 60 mg to about 125 mg, about 60 mg to about 200 mg, about 60 mg to about 300 mg, or about 60 mg to about 400 mg of the 6-(4-Tert-Butylphenoxy)Pyridin-3-Amine monohydrochloride salt. In other embodiments, the composition comprises a unit dose of about 50 mg to about 150 mg, about 100 mg to about 200 mg, about 150 mg to about 250 mg, about 200 mg to about 300 mg, about 250 mg to about 350 mg, or about 300 mg to about 400 mg of the 6-(4-Tert-Butylphenoxy)Pyridin-3-Amine monohydrochloride salt, wherein about 250 mg to about 350 mg is preferred. In a further preferred embodiment the composition comprises a unit dose of about 30 mg to about 300 mg or one of its salts, solvates, tautomers, or stereoisomers thereof, preferably of the 6-(4-Tert-Butylphenoxy)Pyridin-3-Amine monohydrochloride salt In a further preferred embodiment the composition comprises a unit dose of about 60 mg to about 300 mg or one of its salts, solvates, tautomers, or stereoisomers thereof, preferably of the 6-(4-Tert-Butylphenoxy)Pyridin-3-Amine monohydrochloride salt. Most preferred is composition comprising a unit dose of about 250 mg to about 350 mg of the 6-(4-Tert-Butylphenoxy)Pyridin-3-Amine monohydrochloride salt dministered to said subject.

In some embodiments, the pharmaceutical composition comprises a unit dose of about 60, 120, 130, 140, 150, 160, 170, 180, 190, 200, 220, 240, 250, 260, 280, 300, 320, 340, 350, 360, 380, 400, 420, 440, 450, 460, 480, 500, 520, 540, 550, 560, 580, 600, 620, 640, 650, 660, 680, 700, 720, 740, 760, 780, or 800 mg of the 6-(4-Tert-Butylphenoxy)Pyridin-3-Amine monohydrochloride salt. In other embodiments, the pharmaceutical composition comprises a unit dose of about 120, 150, 170, 200, 250, 300, 350, 400, 450, 500, 550, 600, 650, 700, 750, or 800 mg of the 6-(4-Tert-Butylphenoxy)Pyridin-3-Amine monohydrochloride salt. In other embodiments, the pharmaceutical composition comprises a unit dose of about 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, or 690-710 mg of the 6-(4-Tert-Butylphenoxy)Pyridin-3-Amine monohydrochloride salt. In other embodiments, the pharmaceutical composition comprises a unit dose of about120-140, 140-160, 170-180, 200-230, 250-270, 290-320, 340-360, 380-400, 440-460, 470-490, 500-550, 550-570, 590-620, 640-660, or 690-710 mg of the 6-(4-Tert-Butylphenoxy)Pyridin-3-Amine monohydrochloride salt. In other embodiments, the pharmaceutical composition comprises a unit dose of about 120-140, 140-160, 160-170, 170-180, 190-200, 200-230, 230-250, 250-270, 270-290, 290-320, 320-340, 340-360, 360-380, 380-400, 400-420, 420-440, 440-460, 460-470, 470-490, 490-500, 500-550, 550-570, 570-590, 590-620, 620-640, 640-660, 660-690, 690-710, 710-740, 740-770, 770-800, 800-830, 830-860, 860-890, 890-920, 920-950, 950-980, 980-1010, 1010-1040, 1040-1070, 1070-1100, 1100-1130, 1130-1160, or 1160-1200 mg of the 6-(4-Tert-Butylphenoxy)Pyridin-3-Amine monohydrochloride salt. In other embodiments, the pharmaceutical composition comprises a unit dose of about 120-140, 140-160, 170-180, 200-230, 250-270, 290-320, 340-360, 380-400, 420-440, 460-470, 490-500, 550-570, 590-620, 640-660, 690-710, 740-770, 800-830, 860-890, 920-950, 980-1010, 1040-1070, 1100-1130, or 1160-1200 mg of the 6-(4-Tert-Butylphenoxy)Pyridin-3-Amine monohydrochloride salt.In other embodiments, the pharmaceutical composition comprises a unit dose about 60, 120, 170, 250, 400, or 600 mg.

Normally, the composition is administered orally as a solid or liquid dosage form. Alternatively, the composition can be a lyophilized powder to be reconstituted before use.

The cancers and/or tumors to be treated with the pharmaceutical composition of the present invention are as described supra.

### EXAMPLES

The invention will now be further described by means of the following non-limiting examples.

### Example 1—Phase I/IIA Clinical Study of 6-(4-Tert-Butylphenoxy)Pyridin-3-Amine in Adult Patients With Locally Advanced or Metastatic Solid Tumour Malignancies

This Phase IIIA study is an ongoing first-in-human, open-label study investigating the safety, tolerability, pharmacokinetics (PK), pharmacodynamics (PD) and preliminary efficacy of the pan-NOTCH inhibitor 6-(4-Tert-Butylphenoxy)Pyridin-3-Amine in adult patients with locally advanced or metastatic solid tumours or hematologic malignancies.

6-(4-Tert-Butylphenoxy)Pyridin-3-Amine is orally administered and is a small molecule targeting the NOTCH signalling pathway with binding to the NOTCH-specific transcription complex located at the nucleus of cells. The study is divided into two parts with Part A being the dose escalation part of the study to determine the maximum tolerated dose (MTD) or recommended Phase II dose (RP2D). In Part A at least 3, up to 6 patients per dose will participate in the sequentially enrolled dose cohorts. In an additional cohort of approximately 45-50 patients, the safety of the previously determined RP2D will be confirmed (dose confirmatory cohort).

Part B is the expansion part of the study with several expansion arms to confirm MTD/RP2D of 6-(4-Tert-Butylphenoxy)Pyridin-3-Amine in patients with selected tumour indications and to explore preliminary clinical efficacy and PD of 6-(4-Tert-Butylphenoxy)Pyridin-3-Amine in these indications. In all expansion arms in Part B all patients have tumours characterised by a functionally over-activated NOTCH signalling pathway determined by molecular and/or biochemical biomarkers. With 10-20 patients enrolled into each expansion arm it is estimated that about 100-120 patients will need to be recruited for part B.

The study is designed as an open label, non-randomised, uncontrolled Phase IIIA dose escalation study with expansion cohorts of 6-(4-Tert-Butylphenoxy)Pyridin-3-Amine administered orally on a once-daily schedule, based on a 28-day treatment cycle. The dosing will be 15, 30, 60, 120, 170, 250, 400, or 600 mg of the 6-(4-Tert-Butylphenoxy)Pyridin-3-Amine monohydrochloride salt or higher per day or alternatively 20, 40, 60, 120, 170, 250, 400, or 600 mg of the 6-(4-Tert-Butylphenoxy)Pyridin-3-Amine monohydrochloride salt or higher per day. Dosing of 15, 30, 60, 120, 170, 250, 400, or 600 mg of the 6-(4-Tert-Butylphenoxy)Pyridin-3-Amine monohydrochloride salt corresponds to dosing of 13, 26, 52, 104, 148, 217, 348, or 522 mg of 6-(4-Tert-Butylphenoxy)Pyridin-3-Amine (free base). Dosing of 20, 40, 60, 120, 170, 250, 400, or 600 mg of the 6-(4-Tert-Butylphenoxy)Pyridin-3-Amine monohydrochloride salt corresponds to dosing of 17, 35, 52, 104, 148, 217, 348, or 522 mg of 6-(4-Tert-Butylphenoxy)Pyridin-3-Amine (free base). For this study, 6-(4-Tert-Butylphenoxy)Pyridin-3-Amine monohydrochloride salt was formulated into a capsule having a drug load of 5, 10, 20, 50 and 100 mg, respectively intended to be administered orally.
Patients will meet the following criteria:

Histologically or cytologically confirmed solid tumours that are surgically unresectable, locally advanced, or metastatic which have progressed on at least one line of systemic therapy (with the exception of adenoid cystic carcinoma patients who are allowed to be systemic treatment-naïve) and for which no standard-of-care therapy exists.
OR
Relapsed or refractory (r/r) T-cell acute lymphoblastic leukaemia (T-ALL) or lymphoma (T-LBL). Refractory patients are defined as T-ALL/T-LBL patients with ≥5% bone marrow blasts, and/or concomitant extramedullary involvement, who have not achieved a CR after standard induction/consolidation therapy attempt. Relapsed patients are defined as T-ALL/T-LBL patients who have recurrent disease, i.e. ≥5% bone marrow blasts and/or concomitant extramedullary relapse, after having achieved a prior CR.

For Part A (dose-escalation) solid tumour indications based on known involvement of the NOTCH pathway activation in these indications such as Adenoid cystic carcinoma (ACC), Triple negative breast cancer (TNBC), Breast cancer (ER+/−and HER2+/−), Prostate cancer, Osteosarcoma, Malignant glomus tumour, colorectal cancer in patients resistant to oxaliplatin or irinotecan-based therapy or hepatocellular carcinoma.

For Part A (MTD/RP2D confirmatory cohort) ACC and breast cancer (TNBC, ER+/−, HER2+/−) and T-ALL/T-LBL with confirmed Notch pathway activation: Additionally any other cancer (haematologic or solid) with a confirmed NOTCH pathway activation, may be included.

Patients with solid tumours must have at least one measurable lesion (at least 1.0 cm in diameter) according to Response Evaluation Criteria in Solid Tumours (RECIST) v1.1 guideline for solid tumours (irradiated lesions are only measurable if unequivocal disease progression is demonstrated).

Patients in the MTD/RP2D confirmatory cohort of Part A and all patients in Part B: Patients must have tumours characterised by activation of the NOTCH signalling pathway (either by mutations, amplification/translocations or gene/protein expression alteration) validated by molecular and/or biochemical biomarkers assessed by using established laboratory methods. Patients must have tumour lesions accessible to biopsy.

### Demography

a. Men and women≥18 years old on the day of signing informed consent.
b. Eastern Cooperative Oncology Group (ECOG) performance status 0 or 1.

Patients must have the following laboratory values:
1. Total serum bilirubin≤1.5×upper limit of normal (ULN)
2. Alkaline phosphatase (ALP)≤2.5× ULN; if liver function abnormalities are due to the underlying malignancy and known bone metastases, then ALP must be≤5×ULN
3. Serum aspartate aminotransferase (AST/SGOT) and alanine aminotransferase (ALT/SGPT)≤2.5× ULN; if liver function abnormalities are due to the underlying malignancy and known hepatic metastases or bone metastases, then AST and ALT must be≤5×ULN
4. Serum creatinine≤1.5×ULN; or if serum creatinine≤1.5×ULN, then serum creatinine clearance (CrCl)≥50 mL/min (estimated by Cockcroft-Gault formula)
5. Magnesium, potassium, total calcium levels (corrected for serum albumin), and phosphorus levels within normal limits or correctable with supplements
6. Serum albumin concentration≥30 g/L
7. Patients with solid tumors must have:
   a. Absolute neutrophil count (ANC)≥1.5×10⁹/L
   b. Haemoglobin (Hgb)≥10 g/dL (≥100 g/L)
   c. Platelet count≥75×10⁹/L (without platelet transfusion or growth factor support in the preceding 7 days)
   d. Partial thromboplastin time (PTT)≤1.5×ULN and international normalised ratio (INR)≤1.3 (unless the patient is receiving therapeutic anticoagulants)

Part B of the study will consist of one or several expansion arm(s) with selected solid tumour indications. For the analysis of the expansion arms a Bayesian Hierarchical binomial-design will be applied with enrolment of at least 10 patients into each arm. With 10-20 patients enrolled into each expansion arm it is estimated that about 100-120 patients will need to be recruited. Patients from the MTD/RP2D confirmatory cohort of Part A that qualify for any of the expansion arms will be included into the analyses of part B and will contribute to the total sample size per arm.

The primary outcome measures for this study are:
Part A (Escalation): Dose limiting toxicities (DLT)
Part B (Expansion): Best overall response according to the respective tumour assessment criteria for the different cancer indications.

### Results

Fourty-one patients (median age 55 years, range 25 to 76 years) have been treated with 6-(4-Tert-Butylphenoxy)Pyridin-3-Amine in the dose escalation part of the clinical study at oral doses of 15 mg-600 mg QD (once a day). Preliminary safety data indicates that 6-(4-Tert-Butylphenoxy)Pyridin-3-Amine is well tolerated with 73.3% of adverse events (AEs) being mild (Grade 1) or 21% moderate (Grade 2) in severity, and 5.7% Grade 3. All AEs were evenly distributed over the dose cohorts with no evidence of dose relation. Cumulatively, a total of 352 AEs has occurred in 41 patients since study initiation, of which 132 AEs (37.2%) in 31 patients were considered related to the study drug. The treatment-related treatment-emergent AEs (TEAEs) most commonly reported (≥10% of patients) were nausea (24%), diarrhea (20%), dyspepsia (15%), fatigue (12%), and vision blurred (12%).

The majority of treatment-related TEAEs resolved or were resolving at the time of data cut-off without requiring medications or discontinuation of 6-(4-Tert-Butylphenoxy)Pyridin-3-Amine dosing. The study includes intense ECG monitoring. There was one related AE of tachycardia Grade 1 (in cohort 2). Overall, there was no signal of cardiotoxicity and no QTc prolongations have been reported to date.

No events of skin phototoxicity or eye mydriasis have been reported to date in this study. Of the 352 AEs reported, 12 (3.4%) were considered serious adverse events (SAEs) and included abdominal pain, anorexia, atrial flutter, cardiac failure congestive, drug-induced liver injury, duodenal haemorrhage, dyspnoea, general physical condition decreased, respiratory tract infection, spinal cord compression, transaminase increase, and tumour pain. Of these, one SAE was considered related to study drug (drug-induced liver injury), which was reported in a patient with metastatic adenoid cystic carcinoma with extensive liver involvement, treated at an intermediate dose level.

One patient in cohort 8 (600 mg) experienced one dose limiting toxicity (DLT). This was an elevation of the gamma-glutamyl transferase (GGT), grade 3, that was asymptomatic and deemed not clinically significant by the investigator. There has been one unrelated SAE with fatal outcome due to respiratory tract infection in cohort 1 (15 mg). No deaths due to drug-related AEs have been reported to date.

6-(4-Tert-Butylphenoxy)Pyridin-3-Amine is rapidly absorbed after oral administration and reaches Cmax within 1 to 2 hours of dosing. Plasma exposure increases with increasing doses. There were no signs of saturated absorption in the dose range tested. The elimination half-life is approximately 20 hours and 6-(4-Tert-Butylphenoxy)Pyridin-3-Amine accumulates about 2-fold with repeated daily administration. Steady state concentrations are reached within one week of dosing.

Hair follicles were obtained at pre-defined time points to measure changes in NOTCH target genes expression by 6-(4-Tert-Butylphenoxy)Pyridin-3-Amine treatment. NOTCH target gene down-regulation in hair follicles has been reported to be a robust and indicative biomarker that correlates with both clinical efficacy and the incidence of DLTs (Tanis K Q, et al. Clin Pharmacol Ther 2016; 99(4):370-80; Cook N, et al. BJC 2018; 118(6):793-801).

Between 5 to 10 hairs were plucked to extract the complete and undamaged hairbulb (full hair follicle), and processed as described in Camidge D R, et al. Br J Cancer 2005; 92:1837-41 and Bradley B J, et al. Mol EcolNotes 2005; 5:961-4. RNA concentration was measured by Qubit (RNAHighSensitivity), and RNA integrity was evaluated on a RNA 6000 Pico chip. RNA samples were tested for gene expression profiling on a Nanostring platform.

In addition to hair follicles, gene expression profiling to measure changes in NOTCH target genes was also performed in whole blood as an alternative surrogate tissue.

Whole blood samples for gene expression analysis were collected in PAXgene Blood RNA Tubes and RNA extracted following their standard Blood RNA Kit for silica-membrane-based RNA isolation and purification (Beckton Dickinson). RNA samples were tested for gene expression profiling on a Nanostring platform.

In patients in the ongoing phase 1 study, average target gene downregulation compared to baseline prior to treatment with 6-(4-Tert-Butylphenoxy)Pyridin-3-Amine showed a strong down-regulation of several NOTCH target genes starting at Cycle 2 day 1 (C2D1) and reaching values higher than 90% inhibition, as shown in Table 1 below.

Table 1: NOTCH target down regulation in hair follicles and blood cells from Phase 1 patients. Target gene down regulation (CCND3, cyclin-D3; HES1, Hairy and enhance of split-1; HES4, Hairy and enhance of split-4; HESS, Hairy and enhance of split-5; HEY1, hes related family bHLH transcription factor with YRPW motif1; HEY2; hes related family bHLH transcription factor with YRPW motif 2; HEYL, hes-related family bHLH transcription factor with YRPW motif-like; IL7R-α, Interleukin-7 receptor subunit alpha; NOTCH1; NOTCH2; NRARP (NOTCH Regulated Ankyrin Repeat Protein):

| | Inhibition | | |
|---|---|---|---|
| Dose group | range | Target genes | Matrix |
| 60 mg* | up to 80% | HES4, HES5, NRARP, | Hair Follicle |
| 120 mg* | up to 95% | CCND3, HES4, HES5, | Hair Follicle |
| | | HEYL, HEY2 | |
| 170 mg* | up to 75% | HES1 | Hair Follicle |
| 250 mg* | up to 80% | CCND3, HES1, HEY2, NRARP | Hair Follicle |
| | | CCND3, HEY1, IL7Rα, | |
| | | NOTCH1, | |
| 600 mg* | up to 50% | NOTCH2 | Whole Blood |

*6-(4-Tert-Butylphenoxy)Pyridin-3-Amine monohydrochloride salt

In the dose escalation cohort, patients in dose group (DG) 4 receiving 120mg of 6-(4-Tert-Butylphenoxy)Pyridin-3-Amine monohydrochloride salt and patients in higher dose groups reported visual symptoms with increasing incidence. It is believed that the occurrence of visual symptoms is a pharmacodynamic effect of 6-(4-Tert-Butylphenoxy)Pyridin-3-Amine. This makes a dose of 120 mg and higher of the 6-(4-Tert-Butylphenoxy)Pyridin-3-Amine monohydrochloride salt the therapeutic dose of choice.

TABLE 2
Visual symptoms reported with respect to dose group (DG) of 6-(4-Tert-
Butylphenoxy)Pyridin-3-Amine monohydrochloride salt: DG1 (15 mg), DG2 (30 mg), DG3
(60 mg), DG4 (120 mg), DG5 (170 mg), DG6 (250 mg), DG7 (400 mg), DG7 (600
mg). Visual symptoms were first reported by patients in DG4 receiving 120 mg of 6-(4-Tert-
Butylphenoxy)Pyridin-3-Amine monohydrochloride salt. Visual symptoms were of varying
phenotype. There seemed to be a trend of higher incidence in dose groups receiving 250 mg or
higher doses (DG5 through DG8).

| | | DG1 | DG2 | DG3 | DG4 | DG5 | DG6 | DG7 | DG8 |
|---|---|---|---|---|---|---|---|---|---|
| Total pts treated per DG | | 5 | 3 | 7 | 7 | 3 | 4 | 3 | 9 |
| Eye-related AE (reported Term) | #AEs | n* | n* | n* | n* | n* | n* | n* | n* |
| Adaptative visual difficulty | 1 | | | | | | | 1 | |
| Blurred vision | 6 | | | | 1 | 1 | 1 | | 1 |
| Eye disorder | 2 | | | | | | | | 1 |
| Intermittent color vision deficiency/ | 2 | | | | | | | | 1 |
| Intermittent blurred vision | | | | | | | | | |
| Photophobia | 2 | | | | | | | | |
| Photopsy | 3 | | | | | | 1 | | |
| Transient defect in accommodation | 7 | | | | 2 | 1 | | | 1 |
| to dim light | | | | | | | | | |
| Visual disturbance in light exposure | 1 | | | | | | 1 | | |
| Visual alteration | 2 | | | | | | | 1 | 1 |
| Total | 26 | 0 | 0 | 0 | 3 | 2 | 3 | 2 | 6 |
| % on total pts per DG | — | — | — | — | 43% | 67% | 75% | 67% | 67% |

DG, dose group;
AE, adverse event.

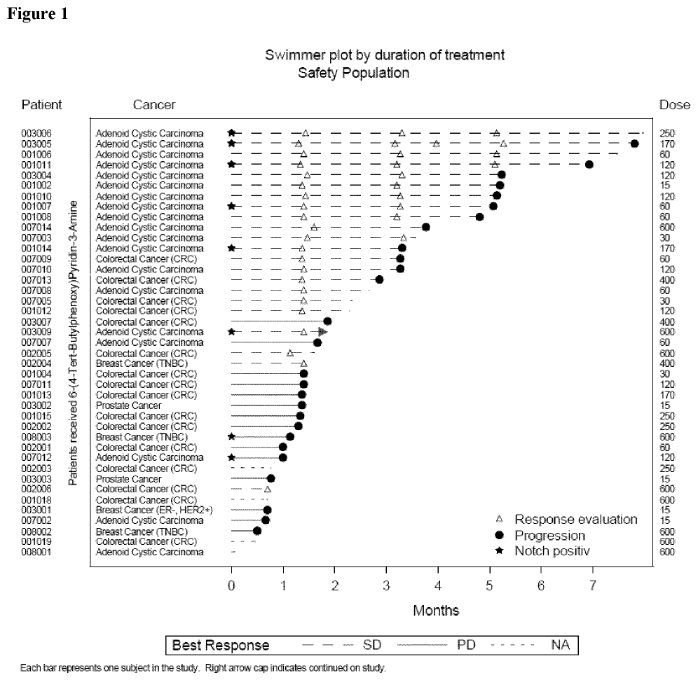

Clinically, best response was durable stable disease (SD) as can be seen from FIG. 1, with no or only mild AE at doses showing target engagement. Ten patients with ACC had radiologically confirmed SD, of whom 4 patients had a Notch activating mutation or fusion. The Notch status of the other patients is under investigation. Disease control rate (DCR) of 19 ACC patients at 8 weeks and 20 weeks was 79% and 58%, respectively (DCR defined as number of patients with CR, PR, or SD divided by the number of patients treated).

### Example 2—Phase IIA Clinical Study of 6-(4-Tert-Butylphenoxy)Pyridin-3-Amine and a Further Active Ingredient in 12-40 Years Old Patients With T-ALL/T-LBL

In a study, 6-(4-Tert-Butylphenoxy)Pyridin-3-Amine and a further active ingredient will be tested in 12-40 years old patients with T-ALL/T-LBL, patients with first or subsequent bone marrow recurrence or refractory to at least one induction attempt may be included. The study is designed as a single arm, open-label, multi-centre, phase II clinical trial to evaluate the efficacy and safety of 500 mg 6-(4-Tert-Butylphenoxy)Pyridin-3-Amine (free base) administered orally on a once-daily schedule in patients with NOTCH positive T-lineage ALL/LBL. Initial treatment with 6-(4-Tert-Butylphenoxy)Pyridin-3-Amine single agent is given for 28 days (one “cycle”). Patients are assessed for disease response on day 29; if residual leukemia or lymphoma is present, a second course of 6-(4-Tert-Butylphenoxy)Pyridin-3-Amine is administered as above. The primary efficacy measure is ORR calculated as the sum of patients achieving a CR or a CRi in patients with T-ALL divided by the total number of patients enrolled in each part of the study,

### Example 3—Compassionate Use Program in a NOTCH Positive Relapsed Refractory T-ALL Patient

In a compassionate use program, a 24 years old patient with Notch-activated relapsed and treatment refractory T-ALL was treated with 6-(4-Tert-Butylphenoxy)Pyridin-3-Amine (free base) once daily starting with 300 mg, increased to 400 mg, then 500 mg in addition to his ongoing anti-cancer treatment. Dose escalation was done every 3 days to reach the target dose 500 mg. Within 1 week after starting dosing of 6-(4-Tert-Butylphenoxy)Pyridin-3-Amine the patient achieved complete remission.

Administration of 6-(4-Tert-Butylphenoxy)Pyridin-3-Amine induced a potent time-dependent downregulation of DTX-1 (Deltex E3 Ubiquitin Ligase 1) up to 95% at C2D15, indicative of NOTCH pathway downregulation. DTX-1 is a positive regulator of NOTCH signalling pathway

6-(4-Tert-Butylphenoxy)Pyridin-3-Amine also induced a potent effect on plasma cytokine levels leading to a strong anti-inflammatory response in patients, downregulation of inflammatory cytokines like IL-6 (interleukin 6) and IFNγ (interferon gamma) up to 95%, and upregulation of anti-inflammatory cytokines like IL-10 (interleukin 10) up to 65% as can be seen from Table 3 below. Since IL6, IFNγ and IL-10 expression is also regulated downstream NOTCH pathway, the effect observed these biomarkers represent further evidence of NOTCH pathway modulation by 6-(4-Tert-Butylphenoxy)Pyridin-3-Amine.

Blood samples for PBMCs and cytokine analysis were collected using BD Vacutainer® Evacuated Blood Collection Tubes, and immediately centrifuged to separate the cell and plasma compartments for further analysis. Gene expression profiling was performed by standard RTqPCR. Cytokine analysis in the plasma compartment was performed with a standard commercially available ELISA method.

TABLE 3
NOTCH target protein down regulation
Plasma Cytokines

| Dose | Change range | Cytokines | Matrix |
|---|---|---|---|
| 500 mg | up to 95% decrease | IL-6, IFNγ | EDTA plasma |
| 500 mg | up to 65% increase | IL-10 | EDTA plasma |

### EMBODIMENTS

1. Method of treating a Notch-dependent cancer in a subject, comprising administering to said subject 6-(4-Tert-Butylphenoxy)Pyridin-3-Amine or one of its salts, solvates, tautomers, or stereoisomers thereof, wherein about 50 mg to about 700 mg of 6-(4-Tert-Butylphenoxy)Pyridin-3-Amine is administered daily to said subject.
2. The method according to embodiment 1, wherein the salt is a hydrochloride salt or a hydrobromide salt.
3. The method according to embodiment 2, wherein the hydrochloride salt is the monohydrochloride salt.
4. The method according to any of the preceding embodiments, wherein the cancer is characterised by activation of the Notch signalling pathway.
5. The method according to any of the preceding embodiments, wherein the cancer is characterised by alterations and/or overactivation of the Notch signalling pathway via mutations, translocations, and/or over-expression.
6. The method according to any of the preceding embodiments, wherein the subject has histologically or cytologically confirmed advanced cancers whose disease has relapsed or progressed upon standard therapy.
7. The method according to any of the preceding embodiments, wherein the subject has a solid tumour.
8. The method according to any of the preceding embodiments, wherein the subject has an advanced and/or metastatic solid tumour.
9. The method according to any of the preceding embodiments, wherein the subject has a cancer selected from the group consisting of adenoid cystic carcinoma (ACC), breast cancer, prostate cancer, osteosarcoma, malignant glomus tumour, colorectal cancer and hepatocellular carcinoma, preferably selected from the group consisting of adenoid cystic carcinoma (ACC), triple negative breast cancer (TNBC), ER+ breast cancer, ER− breast cancer, HER2+ breast cancer, HER2− breast cancer, prostate cancer, osteosarcoma, malignant glomus tumour, colorectal cancer in patients resistant to oxaliplatin or irinotecan-based therapy and hepatocellular carcinoma.
10. The method according to any of the embodiments 1 to 8, wherein the subject has a cancer selected from the group consisting of adenoid cystic carcinoma (ACC), breast cancer, colorectal cancer and prostate cancer.
11. The method according to any of the embodiments 1 to 8, wherein the subject has breast cancer, preferably breast cancer selected from the group consisiting of triple negative breast cancer, ER− breast cancer, and HER2+ breast cancer).
12. The method according to any of the embodiments 1 to 8, wherein the subject has adenoid cystic carcinoma.
13. The method according to any of the embodiments 1 to 8, wherein the subject has malignant glomus tumour.
14. The method according to any of the embodiments 1 to 8, wherein the subject has colorectal cancer, preferably colorectal cancer wherein the subject is resistant to oxaliplatin or irinotecan-based therapy.
15. The method according to any of the embodiments 1 to 8, wherein the subject has prostate cancer.
16. The method according to any of the embodiments 1 to 8, wherein the subject has hepatocellular carcinoma.
17. The method according to any of the embodiments 1 to 8, wherein the subject has sarcoma.
18. The method according to embodiment 17, wherein the subject has osteosarcoma.
19. The method according to embodiment 17, wherein the subject has osteosarcoma, liposarcoma, rhabdomyosarcoma, or fibrosarcoma.
20. The method according to embodiment any of the embodiments 1 to 8, wherein the subject has gastric cancer, cholangiocellular carcinoma, ovarian cancer, cervical cancer, prostate cancer, non-small cell lung cancer, lung adenocarcinoma, melanoma, or glioblastoma multiforme.
21. The method according to any of the embodiments 1 to 6, wherein the subject has haematological malignancies.
22. The method according to any of the embodiments 1 to 6 or 21, wherein the subject has acute lymphoblastic leukemia, T-cell acute lymphoblastic leukemia (T-ALL) or T-cell lymphoblastic lymphoma (T-LBL), acute myeloid leukemia, chronic lymphocytic leukemia, or chronic myelocytic leukemia.
23. The method according to any of the embodiments 1 to 6 or 21, wherein the subject has Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma.
24. The method according to any of the preceding embodiments, comprising daily administration of about 50 mg to about 550 mg of 6-(4-Tert-Butylphenoxy)Pyridin-3-Amine.
25. The method according to any of the preceding embodiments, comprising daily administration of about 100mg to about 700 mg of 6-(4-Tert-Butylphenoxy)Pyridin-3-Amine.
26. The method according to any of the preceding embodiments, comprising daily administration of about 100 mg to about 550 mg of 6-(4-Tert-Butylphenoxy)Pyridin-3-Amine.
27. The method according to any of the preceding embodiments, comprising daily administration of about 400 mg to about 650 mg of 6-(4-Tert-Butylphenoxy)Pyridin-3-Amine.
28. The method according to any of the preceding embodiments, comprising daily administration of about 350 mg to about 550 mg of 6-(4-Tert-Butylphenoxy)Pyridin-3-Amine.
29. The method according to any of the preceding embodiments, comprising daily administration of about 250 mg to about 450 mg or of about 400 mg to about 700 mg of 6-(4-Tert-Butylphenoxy)Pyridin-3-Amine.
30. The method according to any of the preceding embodiments, wherein the dose is about 100-110, 120-140, 140-160, 170-180, 200-230, 250-270, 290-320, 340-360, 380-400, 440-460, 470-490, 500-550, 550-570, 590-620, 640-660, or 690-710 mg.

31. The method according to any of the preceding embodiments, wherein the dose is about 45-55, 100-110, 140-160, 200-230, 340-360, or 500-550 mg per day.
32. The method according to any of the preceding embodiments, wherein 6-(4-Tert-Butylphenoxy)Pyridin-3-Amine or one of its salts, solvates, tautomers, or stereoisomers thereof is administered twice a day.
33. The method according to any of the preceding embodiments, wherein said patient was pre-treated with standard of care.
34. The method according to any of the preceding embodiments, wherein the subject has cancers characterised by activation of the Notch signalling pathway as validated by molecular and/or biochemical biomarkers.
35. 6-(4-Tert-Butylphenoxy)Pyridin-3-Amine or one of its salts, solvates, tautomers, or stereoisomers thereof for use in the treatment of a Notch-dependent cancer in a subject comprising daily administration of about 50 mg to about 700 mg of 6-(4-Tert-Butylphenoxy)Pyridin-3-Amine to said subject.
36. Use of 6-(4-Tert-Butylphenoxy)Pyridin-3-Amine or one of its salts, solvates, tautomers, or stereoisomers thereof, for the manufacture of a medicament for treating a Notch-dependent cancer in a subject comprising daily administration of about 50 mg to about 700 mg of 6-(4-Tert-Butylphenoxy)Pyridin-3-Amine to said subject
37. 6-(4-Tert-Butylphenoxy)Pyridin-3-Amine or one of its salts, solvates, tautomers, or stereoisomers thereof for use or its use according to embodiment 35 or 36, wherein the salt is hydrochloride salt or hydrobromide salt.
38. 6-(4-Tert-Butylphenoxy)Pyridin-3-Amine or one of its salts, solvates, tautomers, or stereoisomers thereof for use or its use according to embodiment 37, wherein the hydrochloride salt is the monohydrochloride salt.
39. 6-(4-Tert-Butylphenoxy)Pyridin-3-Amine or one of its salts, solvates, tautomers, or stereoisomers thereof for use or its use according to any of the embodiments 35 to 38, wherein the cancer is characterised by activation of the Notch signalling pathway.
40. 6-(4-Tert-Butylphenoxy)Pyridin-3-Amine or one of its salts, solvates, tautomers, or stereoisomers thereof for use or its use according to any of the embodiments 35 to 39, wherein the cancer is characterised by alterations and/or overactivation of the Notch signalling pathway via mutations, translocations, and/or over-expression.
41. 6-(4-Tert-Butylphenoxy)Pyridin-3-Amine or one of its salts, solvates, tautomers, or stereoisomers thereof for use or its use according to any of the embodiments 35 to 40, wherein the subject has a solid tumour.
42. 6-(4-Tert-Butylphenoxy)Pyridin-3-Amine or one of its salts, solvates, tautomers, or stereoisomers thereof for use or its use according to any of the embodiments 35 to 41, wherein the subject has an advanced and/or metastatic solid tumour.
43. 6-(4-Tert-Butylphenoxy)Pyridin-3-Amine or one of its salts, solvates, tautomers, or stereoisomers thereof for use or its use according to any of the embodiments 35 to 42, wherein the subject has a cancer selected from the group consisting of adenoid cystic carcinoma (ACC), breast cancer, prostate cancer, osteosarcoma, malignant glomus tumour, colorectal cancer and hepatocellular carcinoma, preferably selected from the group consisting of adenoid cystic carcinoma (ACC), triple negative breast cancer (TNBC), ER+ breast cancer, ER− breast cancer, HER2+ breast cancer, HER2− breast cancer, prostate cancer, osteosarcoma, malignant glomus tumour, colorectal cancer in patients resistant to oxaliplatin or irinotecan-based therapy and hepatocellular carcinoma.
44. 6-(4-Tert-Butylphenoxy)Pyridin-3-Amine or one of its salts, solvates, tautomers, or stereoisomers thereof for use or its use according to any of the embodiments 35 to 42, wherein the subject has a cancer selected from the group consisting of adenoid cystic carcinoma (ACC), breast cancer, colorectal cancer and prostate cancer.
45. 6-(4-Tert-Butylphenoxy)Pyridin-3-Amine or one of its salts, solvates, tautomers, or stereoisomers thereof for use or its use according to any of the embodiments 35 to 42, wherein the subject has breast cancer, preferably breast cancer selected from the group consisiting of triple negative breast cancer, ER-breast cancer, and HER2+ breast cancer).
46. 6-(4-Tert-Butylphenoxy)Pyridin-3-Amine or one of its salts, solvates, tautomers, or stereoisomers thereof for use or its use according to any of the embodiments 35 to 42, wherein the subject has adenoid cystic carcinoma.
47. 6-(4-Tert-Butylphenoxy)Pyridin-3-Amine or one of its salts, solvates, tautomers, or stereoisomers thereof for use or its use according to any of the embodiments 35 to 42, wherein the subject has malignant glomus tumour.
48. 6-(4-Tert-Butylphenoxy)Pyridin-3-Amine or one of its salts, solvates, tautomers, or stereoisomers thereof for use or its use according to any of the embodiments 35 to 42, wherein the subject has colorectal cancer preferably colorectal cancer wherein the subject is resistant to oxaliplatin or irinotecan-based therapy.
49. 6-(4-Tert-Butylphenoxy)Pyridin-3-Amine or one of its salts, solvates, tautomers, or stereoisomers thereof for use or its use according to embodiment 48, wherein the subject has prostate cancer.
50. 6-(4-Tert-Butylphenoxy)Pyridin-3-Amine or one of its salts, solvates, tautomers, or stereoisomers thereof for use or its use according to any of the embodiments 35 to 42, wherein the subject has hepatocellular carcinoma.
51. 6-(4-Tert-Butylphenoxy)Pyridin-3-Amine or one of its salts, solvates, tautomers, or stereoisomers thereof for use or its use according to any of the embodiments 35 to 42, wherein the subject has sarcoma.
52. 6-(4-Tert-Butylphenoxy)Pyridin-3-Amine or one of its salts, solvates, tautomers, or stereoisomers thereof for use or its use according to embodiment 51, wherein the subject has osteosarcoma.
53. 6-(4-Tert-Butylphenoxy)Pyridin-3-Amine or one of its salts, solvates, tautomers, or stereoisomers thereof for use or its use according to embodiment 51, wherein the subject has osteosarcom, liposarcoma, rhabdomyosarcoma, or fibrosarcoma.
54. 6-(4-Tert-Butylphenoxy)Pyridin-3-Amine or one of its salts, solvates, tautomers, or stereoisomers thereof for use or its use according to any of the embodiments 35 to 42, wherein the subject has gastric cancer, cholangiocellular carcinoma, ovarian cancer, cervical cancer, prostate cancer, non-small cell lung cancer, lung adenocarcinoma, melanoma, or glioblastoma multiforme.
55. 6-(4-Tert-Butylphenoxy)Pyridin-3-Amine or one of its salts, solvates, tautomers, or stereoisomers thereof for use or its use according to any of the embodiments 35 to 40, wherein the subject has haematological malignancies.
56. 6-(4-Tert-Butylphenoxy)Pyridin-3-Amine or one of its salts, solvates, tautomers, or stereoisomers thereof for use or its use according to any of the embodiments 35 to 40 or 55, wherein the subject has acute lymphoblastic leukemia, T-cell acute lymphoblastic leukemia (T-ALL) or T-cell lymphoblastic lymphoma (T-LBL) , acute myeloid leukemia, chronic lymphocytic leukemia, or chronic myelocytic leukemia.
57. 6-(4-Tert-Butylphenoxy)Pyridin-3-Amine or one of its salts, solvates, tautomers, or stereoisomers thereof for use or its use according to any of the embodiments 35 to 40 or 55, wherein the subject has Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma.
58. 6-(4-Tert-Butylphenoxy)Pyridin-3-Amine or one of its salts, solvates, tautomers, or stereoisomers thereof for use or its use according to any of the embodiments 35 to 57, comprising daily administration of about 50 mg to about 550 mg of 6-(4-Tert-Butylphenoxy)Pyridin-3-Amine.
59. 6-(4-Tert-Butylphenoxy)Pyridin-3-Amine or one of its salts, solvates, tautomers, or stereoisomers thereof for use or its use according to any of the embodiments 35 to 57, comprising daily administration of about 100 mg to about 700 mg of 6-(4-Tert-Butylphenoxy)Pyridin-3-Amine.
60. 6-(4-Tert-Butylphenoxy)Pyridin-3-Amine or one of its salts, solvates, tautomers, or stereoisomers thereof for use or its use according to any of the embodiments 35 to 57, comprising daily administration of about 100 mg to about 550 mg of 6-(4-Tert-Butylphenoxy)Pyridin-3-Amine.
61. 6-(4-Tert-Butylphenoxy)Pyridin-3-Amine or one of its salts, solvates, tautomers, or stereoisomers thereof for use or its use according to any of the embodiments 35 to 57, comprising daily administration of about 400 mg to about 650 mg of 6-(4-Tert-Butylphenoxy)Pyridin-3-Amine.
62. Use according to any of the embodiments 35 to 57, comprising daily administration of about 250 mg to about 450 mg of 6-(4-Tert-Butylphenoxy)Pyridin-3-Amine.
63. 6-(4-Tert-Butylphenoxy)Pyridin-3-Amine or one of its salts, solvates, tautomers, or stereoisomers thereof for use or its use according to any of the embodiments 35 to 57, comprising daily administration of about 350 mg to about 550 mg of 6-(4-Tert-Butylphenoxy)Pyridin-3-Amine.
64. 6-(4-Tert-Butylphenoxy)Pyridin-3-Amine or one of its salts, solvates, tautomers, or stereoisomers thereof for use or its use according to any of the embodiments 35 to 57, comprising daily administration of about 400 mg to about 700 mg of 6-(4-Tert-Butylphenoxy)Pyridin-3-Amine.
65. 6-(4-Tert-Butylphenoxy)Pyridin-3-Amine or one of its salts, solvates, tautomers, or stereoisomers thereof for use or its use according to any of the embodiments 35 to 57, wherein the dose is about 100-110, 120-140, 140-160, 170-180, 200-230, 250-270, 290-320, 340-360, 380-400, 440-460, 470-490, 500-550, 550-570, 590-620, 640-660, or 690-710 mg.
66. 6-(4-Tert-Butylphenoxy)Pyridin-3-Amine or one of its salts, solvates, tautomers, or stereoisomers thereof for use or its use according to any of the embodiments 35 to 57, wherein the dose is about 45-55, 100-110, 140-160, 200-230, 340-360, or 500-550 mg per day.
67. 6-(4-Tert-Butylphenoxy)Pyridin-3-Amine or one of its salts, solvates, tautomers, or stereoisomers thereof for use or its use according to any of the embodiments 35 to 66, wherein 6-(4-Tert-Butylphenoxy)Pyridin-3-Amine or one of its salts, solvates, tautomers, or stereoisomers thereof is administered twice a day.
68. 6-(4-Tert-Butylphenoxy)Pyridin-3-Amine or one of its salts, solvates, tautomers, or stereoisomers thereof for use or its use according to any of the embodiments 35 to 67, wherein said patient was pre-treated with standard of care.
69. 6-(4-Tert-Butylphenoxy)Pyridin-3-Amine or one of its salts, solvates, tautomers, or stereoisomers thereof for use or its use according to any of the embodiments 35 to 68, wherein the subject has histologically or cytologically confirmed, advanced cancers whose disease has relapsed or progressed upon standard therapy.
70. 6-(4-Tert-Butylphenoxy)Pyridin-3-Amine or one of its salts, solvates, tautomers, or stereoisomers thereof for use or its use according to any of the embodiments 35 to 69, wherein the subject has cancers characterised by activation of the Notch signalling pathway as validated by molecular and/or biochemical biomarkers.
71. Pharmaceutical composition in unit dose comprising 6-(4-Tert-Butylphenoxy)Pyridin-3-Amine or one of its salts, solvates, tautomers, or stereoisomers thereof, wherein the pharmaceutical composition comprises about 50 mg to about 700 mg of 6-(4-Tert-Butylphenoxy)Pyridin-3-Amine, and a pharmaceutically acceptable carrier.
72. Pharmaceutical composition in unit dose comprising 6-(4-Tert-Butylphenoxy)Pyridin-3-Amine or one of its salts, solvates, tautomers, or stereoisomers thereof, wherein the pharmaceutical composition comprises about 50 mg to about 700 mg of 6-(4-Tert-Butylphenoxy)Pyridin-3-Amine, for use in the treatment of a Notch-dependent cancer in a subject, and a pharmaceutically acceptable carrier.
73. Pharmaceutical composition according to embodiment 71 or 72, wherein the salt is hydrochloride salt or hydrobromide salt.
74. Pharmaceutical composition according to embodiment 71 or 72, wherein the hydrochloride salt is the monohydrochloride salt.
75. Pharmaceutical composition according to any of the embodiments 71 to 74, comprising about 100-110, 120-140, 140-160, 170-180, 200-230, 250-270, 290-320, 340-360, 380-400, 440-460, 470-490, 500-550, 550-570, 590-620, 640-660, or 690-710 mg.
76. Pharmaceutical composition according to any of the embodiments 71 to 75, intended to be administered twice a day.
77. Pharmaceutical composition according to any of the embodiments 71 to 76, wherein the composition is administered orally.
78. Pharmaceutical composition according to any of the embodiments 71 to 77, wherein the composition is a solid dosage form.
79. Pharmaceutical composition according to any of the embodiments 71 to 77, wherein the composition is a lyophilized powder for reconstitution.
80. Pharmaceutical composition according to any embodiments 71 to 77, wherein the composition is a liquid dosage form.
81. Pharmaceutical composition according to any of the embodiments 72 to 80, wherein the cancer is characterised by activation of the NOTCH signalling pathway.

82. Pharmaceutical composition according to any of the embodiments 72 to 81, wherein the subject has a solid tumour.

83. Pharmaceutical composition according to any of the embodiments 72 to 82, wherein the subject has an advanced and/or metastatic solid tumour.

84. Pharmaceutical composition according to any of the embodiments 72 to 81, wherein the subject has haematological malignancies.

1-59. (canceled)

60. A method of treating a Notch-dependent cancer in a subject comprising daily administration of about 120 mg to about 1200 mg of the 6-(4-Tert-Butylphenoxy)Pyridin-3-Amine monohydrochloride salt to said subject.

61. The method of treating according to claim 60, wherein the subject has an advanced and/or metastatic solid tumour.

62. The method of treating according to claim 60, wherein the subject has a cancer selected from the group consisting of adenoid cystic carcinoma (ACC), breast cancer, prostate cancer, osteosarcoma, malignant glomus tumour, colorectal cancer and hepatocellular carcinoma.

63. The method of treating according to claim 60, wherein the subject has a cancer selected from the group consisting of adenoid cystic carcinoma (ACC), breast cancer, colorectal cancer and prostate cancer.

64. The method of treating according to claim 60, wherein the subject has osteosarcoma, liposarcoma, rhabdomyosarcoma, fibrosarcoma, gastric cancer, cholangiocellular carcinoma, ovarian cancer, cervical cancer, prostate cancer, non-small cell lung cancer, lung adenocarcinoma, melanoma, or glioblastoma multiforme.

65. The method of treating according to claim 60, wherein the subject has a haematological malignancy.

66. The method of treating according to claim 60, wherein the subject has acute lymphoblastic leukemia, T-cell acute lymphoblastic leukemia (T-ALL) or T-cell lymphoblastic lymphoma (T-LBL), acute myeloid leukemia, chronic lymphocytic leukemia, chronic myelocytic leukemia, Hodgkin lymphoma, non-Hodgkin lymphoma, follicular lymphoma, diffuse large B cell lymphoma, Burkitt lymphoma, marginal zone B-cell lymphoma, splenic marginal zone lymphoma, mantle cell lymphoma, peripheral T-cell lymphoma, anaplastic large cell lymphoma, CNS lymphoma, or multiple myeloma.

67-80. (canceled)

81. The method of treating according to claim 60, comprising daily administration of about 120 mg to about 800 mg of the 6-(4-Tert-Butylphenoxy)Pyridin-3-Amine monohydrochloride salt.

82. The method of treating according claim 60, comprising daily administration of about 600 mg to about 1000 mg of the 6-(4-Tert-Butylphenoxy)Pyridin-3-Amine monohydrochloride salt.

83-88. (canceled)

89. The method of eating according to claim 60, wherein said subject did not receive prior systemic treatment, or was pre-treated with standard of care or any other systemic treatment.

90. A pharmaceutical composition in unit dose comprising 6-(4-Tert-Butylphenoxy)Pyridin-3-Amine or one of its salts, solvates, tautomers, or stereoisomers thereof, wherein the pharmaceutical composition comprises about 120 mg to about 1200 mg of the 6-(4-Tert-Butylphenoxy)Pyridin-3-Amine monohydrochloride salt, and a pharmaceutically acceptable carrier.

91. (canceled)

92. The pharmaceutical composition according to claim 90, wherein the pharmaceutical composition comprises a unit dose of about 170 mg to about 800 mg of the 6-(4-Tert-Butylphenoxy)Pyridin-3-Amine monohydrochloride salt.

93. The pharmaceutical composition according to claim 90, wherein the pharmaceutical composition comprises a unit dose of about 250 mg to about 800 mg of the 6-(4-Tert-Butylphenoxy)Pyridin-3-Amine monohydrochloride salt.

94. The pharmaceutical composition according to claim 90, wherein the pharmaceutical composition comprises a unit dose of about 400 mg to about 800 mg of the 6-(4-Tert-Butylphenoxy)Pyridin-3-Amine monohydrochloride salt.

95. The pharmaceutical composition according to claim 90, wherein the pharmaceutical composition comprises a unit dose of about 600 mg to about 800 mg of the 6-(4-Tert-Butylphenoxy)Pyridin-3-Amine monohydrochloride salt.

96.-99. (canceled)

100. The pharmaceutical composition according to claim 90, wherein the pharmaceutical composition comprises a unit dose of about 200 mg to about 400 mg of the 6-(4-Tert-Butylphenoxy)Pyridin-3-Amine monohydrochloride salt.

101. The pharmaceutical composition according to claim 90, wherein the pharmaceutical composition comprises a unit dose of about 300 mg to about 500 mg of the 6-(4-Tert-Butylphenoxy)Pyridin-3-Amine monohydrochloride salt.

102. (canceled)

103. The pharmaceutical composition according to claim 90, wherein the pharmaceutical composition comprises a unit dose of about 500 mg to about 700 mg of the 6-(4-Tert-Butylphenoxy)Pyridin-3-Amine monohydrochloride salt.

104. (canceled)

105. (canceled)

106. The pharmaceutical composition according to claim 90, wherein the pharmaceutical composition comprises a unit dose of about 600 mg to about 1000 mg of the 6-(4-Tert-Butylphenoxy)Pyridin-3-Amine monohydrochloride salt.

107. The pharmaceutical composition according to claim 90, wherein the pharmaceutical composition comprises a unit dose of about 600 mg to about 1200 mg of the 6-(4-Tert-Butylphenoxy)Pyridin-3-Amine monohydrochloride salt.

108-112. (canceled)
